(19) United States
(12) Patent Application Publication
(10) Pub. No.: US 2024/0271173 A1
(43) Pub. Date: Aug. 15, 2024

(54) METHODS AND SYSTEMS FOR GENERATING BIOLOGICAL MOLECULES

(71) Applicant: MACHINE BIO INC., Fountain Hills, AZ (US)

(72) Inventors: David Marash, Fountain Hills, AZ (US); Joseph Swabeck, Palo Alto, CA (US)

(21) Appl. No.: 18/290,568

(22) PCT Filed: May 13, 2022
(86) PCT No.: PCT/US2022/029285

§ 371 Date: Nov. 14, 2023

Related U.S. Application Data
(60) Provisional application No. 63/188,724, filed on May 14, 2021.

(51) Int. Cl. C12P 21/00

(52) U.S. Cl. CPC C12P 21/00

(57) ABSTRACT

The present disclosure provides methods and systems for generating biological molecules. The methods and systems may comprise use of a porous membrane or a proteoliposome. The present disclosure also provides methods and systems of generating porous membranes and proteolipsomes

### CROSS-REFERENCE

This application claims the benefit of U.S. Provisional Application No. 63/188,724, filed May 14, 2021, which application is incorporated herein by reference

### BACKGROUND

The ability to produce molecules on demand can have significant implications in various fields such as pharmaceuticals and life sciences research. Manufactured biomolecules can contain impurities that can increase the cost of the preparation of the biomolecules as well as the time taken to prepare the biomolecules. In particular, the cost of preparing a protein can mostly be the cost of purifying the protein from the reaction mixture.

### SUMMARY

Recognized herein is the need for improved biological molecule synthesis approaches that may enable higher purity products with less intensive operating conditions. The present disclosure provides methods and systems for generating a biological molecule, such as a polypeptide or a protein. Methods and systems of the present disclosure may enable the formation of a biological molecule at a high purity (e.g., a purity of at least 60%, 70%, 80%, 90%, 95%, 99%, or greater).

In an aspect, the present disclosure provides a method for generating a biological molecule, comprising: (a) providing a chamber comprising a plurality of cell-free precursors of the biological molecule and a proteoliposome, wherein the proteoliposome comprises a pore; (b) using at least a subset of the plurality of cell-free precursors to form the biological molecule; and (c) during or subsequent to (b), translocating at least a portion of the biological molecule through the pore into the proteoliposome.

In some embodiments, the proteoliposome comprises a lipid bilayer. In some embodiments, the lipid bilayer comprises one or more translocon proteins. In some embodiments, the method further comprises (d) removing the proteoliposome from the chamber. In some embodiments, the biological molecule further comprises an N-terminal translocation signal sequence. In some embodiments, subsequent to (c), the N-terminal translocation signal sequence is removed from the biological molecule. In some embodiments, the translocating occurs substantially simultaneously to the forming the biological molecule. In some embodiments, the translocating occurs subsequently to the forming the biological molecule. In some embodiments, the translocating occurs co-translationally. In some embodiments, the biological molecule is a polypeptide. In some embodiments, the polypeptide is a protein, and wherein at least a portion of the protein is formed in the first portion and folded in the second portion. In some embodiments, the chamber is a part of a flow channel. In some embodiments, the cell-free precursors do not comprise the biological molecule. In some embodiments, (c) comprises translocating an entirety of the biological molecule through sad pore and into the proteoliposome subsequent to (b). In some embodiments, (c) is performed during (b). In some embodiments, (c) is performed subsequent to (b).

In another aspect, the present disclosure provides a system for generating a biological molecule, comprising: a chamber comprising a first portion configured to comprise a plurality of cell-free precursors of the biological molecule and a proteoliposome comprising one or more translocon proteins.

In some embodiments, the proteoliposome comprises hydrophilic polysulfone, mesoporous silica, or mesoporous alumina. In some embodiments, the hydrophilic polysulfone has a molecular weight cut off of at most about 100 kilodaltons. In some embodiments, the one or more translocon proteins comprise one or more proteins selected from the group consisting of SecYEG, SecY, SecE, SecG, Sec61p, and an injectosome. In some embodiments, the plurality of cell-free precursors do not comprise one or more cells. In some embodiments, the plurality of cell-free precursors comprises deoxyribonucleic acid (DNA). In some embodiments, the DNA encodes for the biological molecule. In some embodiments, the biological molecule is a protein, and wherein the proteoliposome comprises conditions for optimal folding of the protein. In some embodiments, the biological molecule is a nucleic acid molecule, a protein, an antigen, a polypeptide, an enzyme, or a chemical. In some embodiments, the proteoliposome comprises one or more signal peptidase proteins.

In another aspect, the present disclosure provides a method for generating a polypeptide, comprising (a) using a cell-free solution comprising a deoxyribonucleic acid molecule encoding the polypeptide to generate a ribonucleic acid molecule, (b) using the ribonucleic acid molecule to generate the polypeptide, and (c) directing the polypeptide through a pore disposed in a proteoliposome.

In some embodiments, subsequent to (c), the polypeptide is present at a purity of at least 60%. In some embodiments, (a)-(c) is performed in a time period of at most 1 day. In some embodiments, the polypeptide comprises a non-native N-terminal signal sequence. In some embodiments, the proteoliposome comprises one or more translocon proteins. In some embodiments, the proteoliposome comprises one or more signal peptidase proteins. In some embodiments, the polypeptide is a protein.

In another aspect, the present disclosure provides a cell-free biological molecule reaction system with a membrane comprising translocon and/or signal peptidase proteins. The translocon proteins can provide a selective channel that permits movement of biological molecules synthesized in a cell-free reaction solution through a membrane while not permitting movement of impurity molecules. The signal peptidase molecules can cleave signal regions from the biological molecules to release the molecules from the membrane and allow the molecules to be collected. This membrane-based system can generate biological molecules at a significantly higher purity as compared to cell-free synthesis alone and can provide new reaction engineering conditions due to the presence of two reaction zones.

In another aspect, the present disclosure provides a method for generating a biological molecule, comprising: (a) providing a chamber comprising a first portion comprising a plurality of cell-free precursors of said biological molecule, a second portion, and a membrane separating said first portion from said second portion, wherein said membrane comprises a pore; (b) using at least a subset of said plurality of cell-free precursors from said first portion to form said biological molecule; and (c) during or subsequent to (b), translocating at least a portion of said biological molecule through said pore into said second portion.

In some embodiments, said membrane comprises a lipid bilayer. In some embodiments, said lipid bilayer is a supported lipid bilayer. In some embodiments, said lipid bilayer comprises one or more translocon proteins. In some embodiments, the method further comprises (d) removing said biological molecule from said second portion of said chamber. In some embodiments, said removing comprises at most about two purification operations. In some embodiments, said removing does not comprise a purification operation. In some embodiments, said biological molecule further comprises an N-terminal translocation signal sequence. In some embodiments, subsequent to (c), said N-terminal translocation signal sequence is removed from said biological molecule. In some embodiments, said translocating occurs substantially simultaneously to said forming said biological molecule. In some embodiments, said translocating occurs subsequently to said forming said biological molecule. In some embodiments, said translocating occurs co-translationally. In some embodiments, said biological molecule is a polypeptide. In some embodiments, said polypeptide is a protein, and wherein at least a portion of said protein is formed in said first portion and folded in said second portion. In some embodiments, said pore has a cross section that is larger than a cross section of said biological molecule. In some embodiments, said chamber is a part of a flow channel. In some embodiments, said cell-free precursors do not comprise said biological molecule. In some embodiments, (c) comprises translocating an entirety of said biological molecule through sad pore and into said second portion subsequent to (b). In some embodiments, (c) is performed during (b). In some embodiments, (c) is performed subsequent to (b).

In another aspect, the present disclosure provides a system for generating a biological molecule, comprising: a chamber comprising a first portion configured to comprise a plurality of cell-free precursors of said biological molecule; a second portion; and a porous membrane separating said first portion from said second portion, wherein said porous membrane comprises a lipid bilayer, and wherein said lipid bilayer comprises one or more translocon proteins.

In some embodiments, said lipid bilayer is a supported lipid bilayer. In some embodiments, said porous membrane comprises hydrophilic polysulfone, mesoporous silica, or mesoporous alumina. In some embodiments, said hydrophilic polysulfone has a molecular weight cut off of at most about 100 kilodaltons. In some embodiments, said one or more translocon proteins comprise one or more proteins selected from the group consisting of SecYEG, SecY, SecE, SecG, Sec61p, and an injectosome. In some embodiments, said plurality of cell-free precursors do not comprise one or more cells. In some embodiments, said plurality of cell-free precursors comprises deoxyribonucleic acid (DNA). In some embodiments, said DNA encodes for said biological molecule. In some embodiments, said biological molecule is a protein, and wherein said second portion comprises conditions for optimal folding of said protein. In some embodiments, said biological molecule is a nucleic acid molecule, a protein, an antigen, a polypeptide, an enzyme, or a chemical. In some embodiments, said supported lipid bilayer comprises one or more signal peptidase proteins.

In another aspect, the present disclosure provides a method for generating a cell-free synthesis chamber, comprising: (a) providing a chamber comprising a first portion and a second portion, wherein said first portion and said second portion are separated by a porous membrane; (b) applying a solution comprising a plurality of proteoliposomes, wherein said plurality of proteoliposomes comprise a lipid bilayer and one or more translocon proteins; and (c) reacting said plurality of proteoliposomes with said porous membrane, wherein said reacting comprises dissociation of said plurality of proteoliposomes to form a lipid bilayer on said porous membrane, wherein said lipid bilayer comprises said one or more translocon proteins.

In some embodiments, said lipid bilayer is a supported lipid bilayer. In some embodiments, said solution comprises a plurality of liposomes without said one or more translocon proteins. In some embodiments, a concentration of said one or more translocon proteins is controlled by a ratio of said proteoliposomes to said plurality of liposomes. In some embodiments, said proteoliposomes are substantially homogenous in size. In some embodiments, said proteoliposomes are generated by incubation of liposomes with cell-free precursors of said translocon proteins.

In another aspect, the present disclosure provides a method for generating a polypeptide, comprising (a) using a cell-free solution comprising a deoxyribonucleic acid molecule encoding said polypeptide to generate a ribonucleic acid molecule, (b) using said ribonucleic acid molecule to generate said polypeptide, and (c) directing said polypeptide through a pore disposed in a membrane.

In some embodiments, subsequent to (c), said polypeptide is present at a purity of at least 60%. In some embodiments, (a)-(c) is performed in a time period of at most 1 day. In some embodiments, said membrane is not a part of a micelle. In some embodiments, said membrane is planer. In some embodiments, said polypeptide comprises a non-native N-terminal signal sequence. In some embodiments, said pore comprises one or more translocon proteins. In some embodiments, said membrane comprises one or more signal peptidase proteins. In some embodiments, said polypeptide is a protein.

In another aspect, the present disclosure provides a system for generating a biological molecule, comprising: a chamber comprising a first portion configured to comprise a plurality of cell-free precursors of said biological molecule; a second portion; and a porous membrane separating said first portion from said second portion, wherein said porous membrane comprises a lipid bilayer, and wherein said lipid bilayer comprises one or more signal peptidase proteins.

In some embodiments, said one or more signal peptidase proteins comprise LepB. In some embodiments, said lipid bilayer further comprises one or more translocon proteins. In some embodiments, said lipid bilayer is a supported lipid bilayer.

In another aspect, the present disclosure provides a system for generating a biological molecule, comprising: a chamber comprising a first portion configured to comprise a plurality of cell-free precursors of said biological molecule, a second portion, and a membrane separating said first portion from said second portion, wherein said membrane comprises a pore; a controller comprising one or more computer processors that are individually or collective configured to direct a method for generating said biological molecule, said method comprising: (i) using at least a subset of said plurality of cell-free precursors from said first portion to form said biological molecule; and (ii) during or subsequent to (i), translocating at least a portion of said biological molecule through said pore into said second portion.

In some embodiments, said method further comprises removing said biological molecule from said second portion of said chamber. In some embodiments, said method further comprises removing an N-terminal translocation signal sequence. In some embodiments, said translocating occurs substantially simultaneously to said forming said biological molecule. In some embodiments, said translocating occurs subsequently to said forming said biological molecule. In some embodiments, said translocating occurs co-translationally.

Another aspect of the present disclosure provides a non-transitory computer readable medium comprising machine executable code that, upon execution by one or more computer processors, implements any of the methods above or elsewhere herein.

Another aspect of the present disclosure provides a system comprising one or more computer processors and computer memory coupled thereto. The computer memory comprises machine executable code that, upon execution by the one or more computer processors, implements any of the methods above or elsewhere herein.

Additional aspects and advantages of the present disclosure will become readily apparent to those skilled in this art from the following detailed description, wherein only illustrative embodiments of the present disclosure are shown and described. As will be realized, the present disclosure is capable of other and different embodiments, and its several details are capable of modifications in various obvious respects, all without departing from the disclosure. Accordingly, the drawings and description are to be regarded as illustrative in nature, and not as restrictive.

### INCORPORATION BY REFERENCE

All publications, patents, and patent applications mentioned in this specification are herein incorporated by reference to the same extent as if each individual publication, patent, or patent application was specifically and individually indicated to be incorporated by reference. To the extent publications and patents or patent applications incorporated by reference contradict the disclosure contained in the specification, the specification is intended to supersede and/or take precedence over any such contradictory material.

### BRIEF DESCRIPTION OF THE DRAWINGS

The novel features of the invention are set forth with particularity in the appended claims. A better understanding of the features and advantages of the present invention will be obtained by reference to the following detailed description that sets forth illustrative embodiments, in which the principles of the invention are utilized, and the accompanying drawings (also “Figure” and “FIG.” herein), of which:

FIG. 1 is a schematic of an example process for generating a biological molecule.

FIG. 2 is an example of a flowchart for a process for generating a cell-free synthesis chamber.

FIG. 3 is an example flowchart for a process for generating a polypeptide.

FIGS. 4A-4D are examples of a method for generating a flow cell chamber

FIG. 5 is an example of a supported lipid bilayer comprising proteins.

FIGS. 6A-6B are examples of a process for generating a chamber comprising a membrane and using the chamber to generate a biological molecule.

FIGS. 7A-7C are examples of a process for generating a biological molecule.

FIG. 8 shows a computer system that is programmed or otherwise configured to implement methods provided herein.

FIG. 9 shows an example of a reactor system configured to produce low to no impurity biological molecules

FIG. 10 shows an example of a liposomes configured for the production of a biological molecule

FIG. 11 shows an example of a discovery platform.

FIG. 12 shows an example of a proteoliposome being ruptured on a porous substrate to release the biological molecule in a reactor.

FIG. 13 shows an example of a biological molecule traversing a pore in supported lipid bilayer.

### DETAILED DESCRIPTION

While various embodiments of the invention have been shown and described herein, it will be obvious to those skilled in the art that such embodiments are provided by way of example only. Numerous variations, changes, and substitutions may occur to those skilled in the art without departing from the invention. It should be understood that various alternatives to the embodiments of the invention described herein may be employed.

Whenever the term “at least,” “greater than,” or “greater than or equal to” precedes the first numerical value in a series of two or more numerical values, the term “at least,” “greater than” or “greater than or equal to” applies to each of the numerical values in that series of numerical values. For example, greater than or equal to 1, 2, or 3 is equivalent to greater than or equal to 1, greater than or equal to 2, or greater than or equal to 3.

Whenever the term “no more than,” “less than,” or “less than or equal to” precedes the first numerical value in a series of two or more numerical values, the term “no more than,” “less than,” or “less than or equal to” applies to each of the numerical values in that series of numerical values. For example, less than or equal to 3, 2, or 1 is equivalent to less than or equal to 3, less than or equal to 2, or less than or equal to 1.

The term “pore,” as used herein, generally refers to a channel or conduit capable of permitting a substance to move from one location to another location. The pore may have at least one opening. In some examples, the pore has at least two openings. The pore can have a cross-section (e.g., diameter) that is on the micrometer or nanometer scale. The pore can have a cross-section that is at most 1 micrometer (um), 500 nanometers (nm), 400 nm, 300 nm, 200 nm, 100 nm, or smaller. The cross-section can be sized to be larger than a longest cross-section of a biological molecule (e.g., polypeptide or protein) to be formed. The pore can be a nanopore (e.g., a pore having a cross-section that is at most 1 um). The pore can be part of a biological molecule, such as a protein (e.g., alpha-hemolysin, a translocon protein, etc.) (e.g., a biological material comprising a pore embedded in a lipid bilayer), or part of a solid-state material, such as, for example, a dielectric (e.g., the pore may be formed within the dielectric), or a combination thereof (e.g., a biological molecule comprising a pore can be positioned over a pore in a solid-state material).

The term “polypeptide,” as used herein, generally refers to a biological molecule comprising at least two amino acids. The polypeptide can be a protein.

The term “cell-free,” as used herein, generally refers to a material that is external to a cell. A cell-free material may be released from the cell, such as, for example, upon lysis or permeabilization of a cell. The cell-free material may have been generated in an environment external to the cell (e.g., generated in a reactor, generated by external proteins of a cell, etc). The cell-free material may be provided or generated in a cell-free environment in which one or more components of the cell (e.g., intracellular components, such as, for example, enzymes, ribosomes, etc.) are present.

In an aspect, the present disclosure provides a method for generating a biological molecule. The biological molecule may be a polypeptide or a nucleic acid molecule, for example. The biological molecule may be a polypeptide (e.g., protein). The biological molecule may be a nucleic acid molecule, such as a deoxyribonucleic acid (DNA) or ribonucleic acid (RNA) molecule.

A method for generating a biological molecule may comprise providing a chamber comprising a first portion containing a plurality of cell-free precursors of the biological molecule, a second portion, and a membrane separating the first portion from the second portion. The membrane may comprise a pore. At least a subset of the plurality of cell-free precursors may be used from the first portion to form the biological molecule. The biological molecule may be translocated through the pore into the second portion.

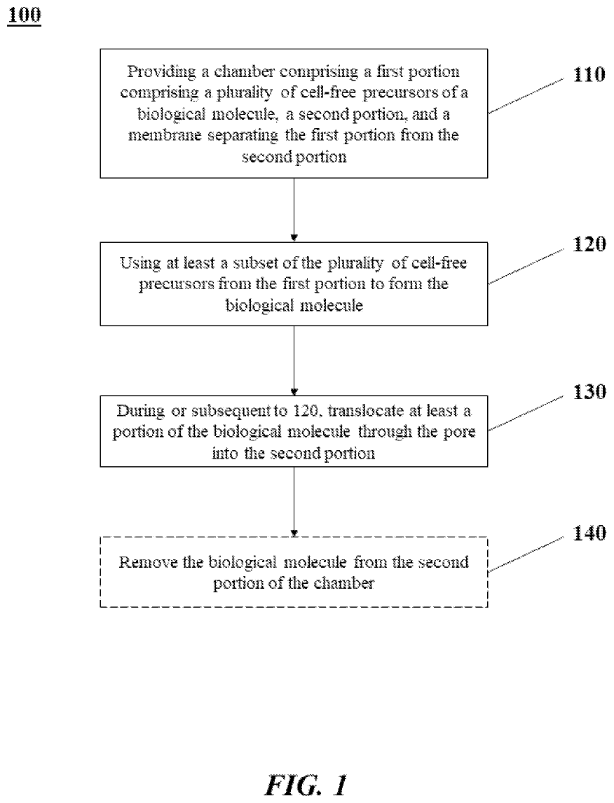

FIG. 1 is a schematic of an example process 100 for generating a biological molecule. In an operation 110, the process 100 may comprise providing a chamber comprising a first portion comprising a plurality of cell-free precursors of a biological molecule, a second portion, and a membrane separating the first portion from the second portion.

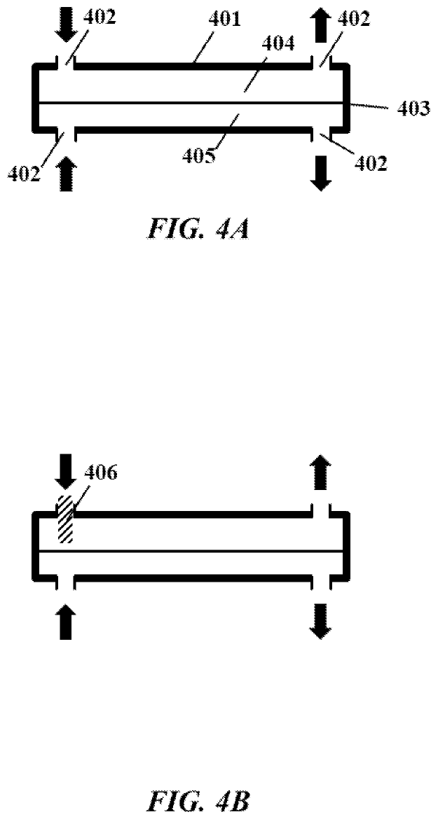

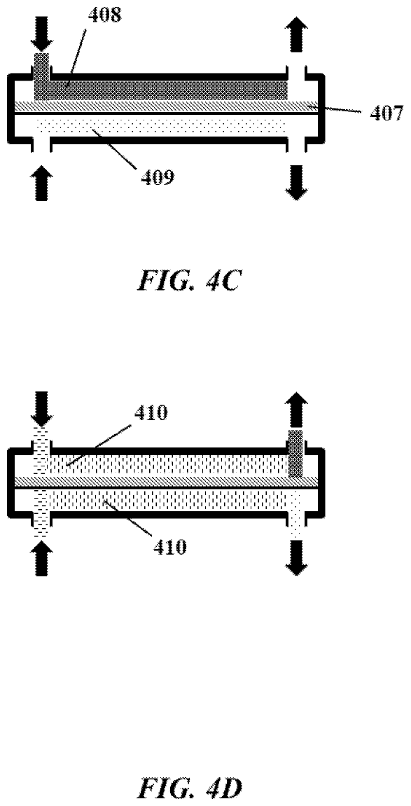

The chamber may be formed of plastic (e.g., polyethylene, polystyrene, resin, polytetrafluoroethylene, etc.), metal (e.g., aluminum, iron, copper), fiber-based materials (e.g., carbon fiber, etc.), or the like, or any combination thereof. The chamber may comprise a plurality of portions. The chamber may comprise at least about 2, 3, 4, 5, 6, 7, 8, 9, 10, or more portions. The chamber may comprise at most about 10, 9, 8, 7, 6, 5, 4, 3, or fewer portions. The chamber may be configured with environmental control apparatuses (e.g., temperature controllers, pressure controllers, etc.), monitoring apparatuses (e.g., thermocouples, pH meters, optical spectroscopy instruments, etc.), electrodes, or the like, or any combination thereof. The chamber may be a part of a flow channel. For example, the chamber may be a part of a flow channel as shown in FIGS. 4A-4D. In some cases, the chamber may not comprise a porous membrane. For example, instead of the chamber being configured to contain a supported lipid bilayer, the chamber can instead be configured with an unsupported lipid bilayer. The chamber may comprise a droplet microfluidic system. For example, the chamber may be a flow chamber configured to separate individual droplets comprising cell-free precursor solutions. The chamber may comprise a plurality of wells. The plurality of wells may be configured to contain a plurality of lipid bilayers. The plurality of wells may be configured such that upon raising a fluid level in the plurality of wells, the plurality of lipid bilayers can be brought into contact to form a single lipid bilayer for use as described elsewhere herein.

A plurality of chambers may be coupled together in series or in parallel. For example, a plurality of chambers can be connected in parallel to improve the throughput of generating biological molecules. In another example, a plurality of chambers can be connected in series, where the product of a first chamber can be used as a reagent in a second chamber. In this example, the biological molecule may be post-translationally modified or incorporated into a biofunctionalized scaffold. The plurality of chambers coupled together in series may be configured such that later chambers comprise one or more analysis instruments configured to analyze the biological molecule. In this way, the plurality of chambers may be configured as a lab-on-a-chip. Subsequent chambers of the plurality of chambers may be configured to biofunctionalize the biological molecule, conjugate bioactive elements to the biological molecule, or the like, or any combination thereof. The biological molecule may be analyzed by co-expression of analysis biological molecules in the first portion of the chamber, and subsequent reaction of the biological molecule with the analysis biological molecules. For example, multiple DNA templates can be expressed at the same time in the first portion, resulting in a plurality of different proteins that can translocate to the second portion and react to form a detectable complex.

The flow channel chamber may comprise a hollow fiber reaction chamber. For example, the chamber may be a hollow fiber configured with translocon proteins within the walls of the fiber configured to remove biological molecules from the fiber. The flow channel may terminate in a dead-end chamber. The dead-end chamber may be configured to accumulate biological molecules for removal. The dead-end chamber may be configured with one or more analysis instruments as described elsewhere herein. The dead-end chamber may comprise a removable chamber. The removable chamber may be a spin plate, a spin column, a filtered chamber, or the like, or any combination thereof.

The membrane may comprise a supported lipid bilayer. The supported lipid bilayer may be supported on the membrane. For example, a supported lipid bilayer can be formed on the membrane. In this example, the supported lipid bilayer can traverse a pore in the membrane. The supported lipid bilayer may comprise one or more molecules comprising a hydrophilic head and a hydrophobic tail. Examples of molecules that may form the supported lipid bilayer include, but are not limited to, phospholipids (e.g., phosphatidylcholines, 1-palmitoyl-2-oleoyl-sn-glycero-3-phosphocholine, dipalmitoylphosphatidylcholine), substituted phospholipids (e.g., phospholipids with one or more substituent groups), fatty acids (e.g., carboxylic acids), prenols, sterols, saccharolipids, polyketides, glycerolipids, sphingolipids, other lipids, or the like, or any combination thereof. The membrane may comprise a pore. The pore may be a protein within the supported lipid bilayer. For example, a transmembrane protein can be positioned above a pore in the membrane to form a pore configured to permit translocation of the biological molecule from the first portion to the second portion. The pore may have a cross section that is larger than a cross section of the biological molecule. The pore may have a cross section that is larger than a cross section of a denatured conformation of the biological molecule. For example, the pore can be large enough to permit a denatured protein to traverse the pore. The pore may have a cross section larger than a cross section of a homo-oligomeric complex or a hetero-oligomeric complex. The homo- or hetero-oligomeric complex may be a complex formed by an oligomerization of the biological molecule. For example, a polypeptide can oligomerize with other polypeptides, and the pore can have a cross section larger than the oligomer.

The supported lipid bilayer may comprise one or more proteins. The one or more proteins may be configured to translocate the biological molecule across the membrane. For example, the biological molecule can pass through a pore in the membrane formed by a protein. The one or more proteins may comprise one or more translocon proteins. For example, the one or more translocon proteins may comprise SecYEG. In another example, the one or more translocon proteins may comprise Sec61p. The one or more proteins may comprise one or more injectosomes. The one or more proteins may comprise one or more hemolysins (e.g., alpha-hemolysin). The one or more proteins may comprise one or more pore forming toxins. The one or more proteins may comprise one or more signal peptidases, signal peptide hydrolases, adenosine triphosphate sythases, enzymes configured to perform post translational modifications, other chaperones, areolysins (e.g., to perform protein sequencing), or the like, or any combination thereof. The supported lipid bilayer may comprise two or more supported lipid bilayers. The two or more supported lipid bilayers may have different proteins from one another.

In some cases, when proteoliposomes are prepared via incorporation of a purified translocon protein (e.g., SecYEG), SecA can be included in the preparation mixture. The SecA may be a protein configured to aid in driving a product (e.g., a protein) through a SecYEG pore. The SecA may not be a membrane protein (e.g., it may not be bound to the lipid bilayer but instead associated with the SecYEG or lipid bilayer). The proteoliposomes produced with SecA may comprise SecYEG integrated into the bilayer with SecA non-covalently associated to the surface of the proteoliposome. Inclusion of SecA may be for both proteoliposomes generated as ingredients for generation of a supported lipid bilayer or for use as independent proteoliposomes as described elsewhere herein.

In some cases, various reagents can be co-expressed with the biological molecule. For example, a reaction mixture can be configured to simultaneously generate the biological molecule as well as translocation factors. In this way, the reagents may not be generated and purified before the reaction, which can simplify the generation of the biological molecule. Examples of reagents that can be co-expressed include, but are not limited to, translocation factors (e.g., signal recognition particles (e.g., Ffh, Ffs), ftsy, SecA, or the like, or any combination thereof). The co-expression may be from a same nucleic acid molecule as the biological molecule. For example, the same DNA can express both SecA and the biological molecule. The co-expression may be from different nucleic acid molecules. The nucleic acid molecule for the reagent may be introduced to the reaction mixture before the nucleic acid molecule for the biological molecule. The nucleic acid molecule for the reagent may be introduced to the reaction mixture after the nucleic acid molecule for the biological molecule. The nucleic acid molecule for the reagent may be introduced to the reaction mixture at the same time as the nucleic acid molecule for the biological molecule. The nucleic acid molecule corresponding to the reagent can be eliminated during the production of the biological molecule. For example, an exonuclease can be added to eliminate a linear DNA molecule corresponding to a cofactor while a plasmid DNA corresponding to the biological molecule is not eliminated.

The biological molecule may be an antibody, an antibody binding protein, a protein, a macromolecule, an enzyme, a nucleic acid molecule, a carbohydrate, a polypeptide, a chemical, or the like, or any combination thereof. The biological molecule may be a polypeptide. The polypeptide may be at least a portion of a protein. The biological molecule may be a biologically active molecule. The biologically active molecule may be a pharmaceutical molecule. For example, a small molecule therapeutic can be generated in the first portion and purified by translocation to the second portion. In another example, a pharmacologically active antibody can be generated in the first portion and subsequently translocated into the second portion where if undergoes folding to become pharmacologically active. The chemical may be a small molecule, a pharmacologically active protein, a toxin, or the like, or any combination thereof.

The biological molecule may comprise a terminal translocation signal sequence. The terminal translocation signal sequence may be an N-terminal translocation signal sequence. The terminal translocation signal sequence may be configured to enable the biological molecule to translocate through the pore. For example, the terminal translocation signal sequence can be a signal sequence for a natively translocated protein. In this example, the terminal translocation signal sequence can permit movement of the biological molecule through the translocon protein.

In another operation 120, the process 100 may comprise using at least a subset of the plurality of cell-free precursors from the first portion to form the biological molecule. The cell-free precursors may not comprise the biological molecule. For example, the cell-free precursors may comprise components of the biological molecule but not the completed biological molecule. The cell free precursors may comprise at least a portion of a homogenized cell. For example, the cell-free precursors can be a homogenized lysate of a cell. In another example, the cell-free precursors can be a minimum set of purified recombinant proteins. The cell-free precursors may comprise cellular bodies (e.g., organelles). The cell-free precursors may comprise substrates (e.g., peptides, nucleic acids, sugars, etc.). The cell-free precursors may comprise one or more energy sources (e.g., adenosine triphosphate, etc.). The cell-free precursors may comprise one or more nucleic acids (e.g., deoxyribonucleic acid, ribonucleic acid, etc.). The one or more nucleic acids may encode for the biological molecule.

The cell-free precursors may comprise a plurality of different nucleic acid templates. The plurality of nucleic acid templates may be at least about 2, 5, 10, 50, 100, 500, 1,000, 5,000, 10,000, or more nucleic acid templates. The plurality of nucleic acid templates may comprise at most about 10,000, 5,000, 1,000, 500, 100, 50, 10, 5, 3, or less nucleic acid templates. The plurality of different nucleic acid templates may be introduced to the first portion of the chamber at the same time. The plurality of different nucleic acid templates may cause generation of a plurality of different biological molecules. For example, a plurality of different proteins can be formed and translocated through the membrane. In this example, subsequent to the translocation, the proteins can interact with binding moieties that bind target proteins while the other proteins are washed away. In this example, the bound proteins can be eluted and subsequently sequenced or otherwise used. The forming of the biological molecule may comprise use of one or more enzymes (e.g., nucleic acid polymerases for forming a nucleic acid, ribosomes for forming a protein, etc.). For example, an RNA can be fed into a ribosome and translated into a polypeptide using the ribosome. In another example, a DNA molecule can be translated into an RNA molecule using a polymerase.

In another operation 130, the process 100 may comprise, during or subsequent to operation 120, translocating at least a portion of the biological molecule through the pore into the second portion. The translocating may occur substantially simultaneously to the forming of the biological molecule. For example, the biological molecule may be formed and translocated through the pore almost as it is formed. In this example, the biological molecule may change conformation in the second chamber (e.g., a protein biological molecule can fold in the second chamber). The translocating may occur subsequently to the forming of the biological molecule. For example, the biological molecule can be generated in the first portion and subsequently diffuse to the membrane, where it can then traverse to the second portion by being driving by a SecA ATPase. In this example, a concentration of the biological molecule may be built up to increase diffusion into the second portion. In some cases, chaperones may be present in the first portion to maintain the biological molecule in an unfolded state prior to translocation through the membrane. The translocating may occur co-translationally. For example, a plurality of the cell-free precursors can begin generation of the biological molecule, the biological molecule can be moved to a pore in the membrane, and the biological molecule can be translocated directly after formation into the second portion. The translocating may be active translocation (e.g., energy is used to translocate the biological molecule through the pore). For example, the translocation can comprise use of adenosine triphosphate to provide energy for the translocation. In another example, an electric field may be applied to facilitate diffusion of the biological molecule through the pore. The translocating may be passive translocation (e.g., the biological molecule can be translocated in an absence of input energy).

A chaperone may be engineered to chaperone a biological molecule. The chaperone may be engineered such that it can chaperone the biological molecule without any engineering of the biological molecule. For example, a chaperone can be engineered to recognize and maintain a biological molecule in an unfolded state using the native structure of the biological molecule. In this example, the biological molecule can be free of an internal region of the biological molecule engineered to couple to the chaperone that is not natively present in the biological molecule. In another example, a SecB chaperone can be modified to chaperone a biological molecule that is not modified to comprise a SecB complementary region. By engineering the chaperone to bind to the unmodified biological molecule, the biological molecule can be maintained with a predetermined composition and structure and not be modified to enable chaperone binding. Such engineering may permit an increased breadth of biological molecules to be formed, since a chaperone binding region is not engineered into the biological molecule. Further, use of a chaperone to stabilize a biological molecule (e.g., an unfolded protein) can enable a post-translational synthesis scheme. A post-translational synthesis scheme may be as described elsewhere herein. For example, a biological molecule can be generated in the first portion, associate with a chaperone configured to maintain the biological molecule in an unfolded state, and subsequently translocate through a pore to the second portion.

A post-translational scheme may provide faster translocation rates (e.g., post-translational translocation can be greater than about ten times faster than co-translational translocation). A post-translational scheme can permit a temporal separation between the translation of the biological molecule and the translocation of the biological molecule through a membrane. The temporal separation may be at least about 30 seconds, 1 minute (m), 5 m, 10 m, 15 m, 30 m, 1 hour (h), 2 h, or more. The temporal separation may be at most about 2 h, 1 h, 30 m, 15 m, 10 m, 5 m, 1 m, 30 s, or less. The duration of the temporal separation may be related to the stability of the chaperone-biological molecule complex. Such temporal separation can enable the generation of the biological molecule to occur in a reactor spatially separated but fluidically connected to a membrane. For example, a large stir tank reactor can be utilized to house a scaled-up synthesis of a biological molecule and be fluidically connected to a membrane as described elsewhere herein. In this example, the synthesis can occur in the tank where a chaperone complexes to the biological molecule, the chaperone-biological molecule complex can be circulated to the membrane, and the biological molecule can be translocated through the membrane to a second portion as described elsewhere herein.

The chaperone may be re-engineered to complex with the biological molecule. This may be in contrast to engineering the biological molecule to complex with the chaperone. The chaperone may be re-engineered by testing various versions and configurations of the chaperone with the biological molecule to determine which version most effectively complexes with the biological molecule. The versions and configurations may be generated by a user editing a native chaperone. For example, mutations of SecB can be performed to generate different SecB versions that can be subsequently tested for affinity to a biological molecule. The chaperone may be engineered for a particular biological molecule without engaging in physical testing of the variant chaperone. For example, a chaperone can be designed to complex with a biological molecule based on the expected properties of the biological molecule. The chaperone may be engineered for different classes of biological molecules. For example, the chaperone can be engineered to interact with conserved domains of a class of biological molecules. In this example, a single chaperone variant can be used with a variety of different biological molecules.

The chaperone may be contained within the second portion. The second portion chaperone may be modified so as not to introduce an impurity into the second portion (e.g., introduce a chaperone impurity into the pure biological molecule solution). The chaperone may be conjugated to the membrane support layer. For example, the chaperone can comprise a domain configured to conjugate to a species attached to the membrane support (e.g., a protein, an antibody, etc.). The chaperone may be selectively conjugated to the membrane support within pores in the support. For example, the membrane support can comprise conjugation species on the insides of the pores in the support. The chaperone may be conjugated to a separation moiety. The separation moiety may comprise a magnetic bead, a molecular magnet, a binding moiety, an affinity moiety (e.g., a moiety configured to bind to an affinity chromatography substrate), or the like, or any combination thereof. For example, the chaperone can be conjugated to a magnetic bead. The separation moiety can be configured to enable separation of the chaperone from the solution comprising the biological molecule. For example, a magnetic field can be applied to the solution to remove a chaperone conjugated to a magnetic bead. The chaperone can be tethered to the membrane. For example, the chaperone can be expressed in the first portion, translocated through the pore, and not cleaved. In this example, the chaperone can remain tethered to the membrane within the second portion. The chaperone may be engineered to be resistant to signal peptidase proteins. Such engineering may permit the chaperone to remain tethered to the membrane in the presence of a signal peptidase protein. The tethering of the chaperone may permit the presence of the chaperone to assist the conformational change of the biological molecule without introducing a chaperone impurity.

The biological molecule may undergo one or more conformational changes subsequent to translocating through the pore. The biological molecule may undergo one or more folding transformations subsequent to translocating through the pore. For example, a protein biological molecule can be formed in the first portion and folded in the second portion. The conditions within the first and second portions of the chamber may be different. For example, the conditions in the first portion can be optimized for synthesis of the biological molecule, while conditions in the second portion can be optimized for folding or other conformational changes. Examples of conditions include, but are not limited to ionic strength, presence or absence of chaperone molecules, presence or absence of enzymes (e.g., enzymes configured to confer post-translational modifications), or the like, or any combination thereof. The conditions within the second portion may be changed over time. For example, a first set of conditions can be present during the production of a first biological molecule, and a second set of conditions can be present during the production of a second biological molecule. In this example, the first set of conditions can be configured to be optimized for a conformational change of the first biological molecule while the second set of conditions can be configured to be optimized for a conformational change of the second biological molecule. A plurality of different conditions can be present within the second portion. For example, a plurality of second portions can be separated from the first portion with a plurality of membranes. In this example, each of the plurality of second portions can comprise a different set of conditions, and the different conditions can be screened to determine an optimal condition for a conformational change of the biological molecule. Such a plurality of conditions may improve the efficiency of a discovery process by enabling hits that were otherwise ignored due to improper folding conditions to be caught and tested. For example, a biological molecule that does not fold well in condition A but does in condition B can be folded in conditions A and B to determine the best folding conditions. In this example, a user can then synthesize the biological molecule and fold it in condition B for further analysis. The presence of the membrane may improve the level to which the conditions can be different in the first and second portions as compared to a system with a single portion. For example, a system with a single portion may have some ability to tune a folding environment, but such ability may be limited by the affects the tuning has on a transcription environment. In this example, a system with a first and second portion separated by a membrane can have vastly different conditions on each side of the membrane without the conditions interfering with one another. The conditions in the first and/or second portion may be made to be similar to cellular conditions. For example, a collection buffer within the second portion can be given a composition similar to that of the cytoplasm of E. coli or the endoplasmic reticulum of a Chinese hamster ovary (CHO) cell. By correlating the conditions in the first and/or second portions with those of a cell, a process developed using the first and second portions may be easily translated to a process using genetically engineered cells. For example, a biological molecule production process using the first and second portions with conditions similar to the cytoplasm of E. coli can then be implemented in an engineered E. coli system with minimal changes to the product biological molecule. In some cases, autophosphorylation of the biological molecule may be performed by including a phosphorylating reagent (e.g., ATP) in the folding buffer. For example, a biological molecule can translocate through a translocon protein and undergo autophosphorylation upon contact with ATP.

In a proteoliposome based system configured to have folding control of a product translocated into the proteoliposome (e.g., a biological molecule), the proteoliposome can be generated by direct synthesis of a translocon protein complex (e.g., SecYEG) into a liposome. For example, a cell free reaction mixture configured to generate SecYEG can be reacted in the presence of bare liposomes to generate proteoliposomes. In some cases, where pre-synthesized translocon proteins are used to generate the proteoliposomes (e.g., by detergent exchange), the folding control reagents may leak through pores that have been induced in the liposomes. In some cases, the solution outside the liposome can be the same as the solution within the proteoliposome, thereby negating the effect of the pores induced in the proteoliposome.

In some cases, where an optimal folding scheme for a biological molecule comprises use of different folding conditions at different stages, an additional chamber may be supplied to provide those different folding conditions. For example, the biological molecule can be generated and translocated into a first chamber comprising a first set of folding conditions. The first chamber may be connected to a second chamber with a second set of folding conditions. Examples of the connection include, but are not limited to, fluidic connection (e.g., the biological molecule can be flowed from the first chamber to the second chamber). The folding conditions in the second chamber can be controlled by adding a solution to the second chamber that, when mixed with the solution from the first chamber, provides the predetermined second folding conditions. In addition, the second chamber may comprise different immobilized species configured to provide a second folding condition. The residence time in the different chambers may be optimized as well. Additional chambers to provide additional folding conditions may be added as well.

The supported lipid bilayer may comprise one or more signal peptidase proteins. The signal peptidase proteins may be configured to cleave a portion of the biological molecule subsequent to translocation through the pore. For example, a biological molecule can be generated with an N-terminal signal sequence that is cleaved by a signal peptidase. Examples of signal peptidase subunits include, but are not limited to, SPC3P, SPC2P, SPC1P, SEC11, SPC12, SPC18, SPC21, SPC22/23 and SPC25. Examples of signal peptidases include, but are not limited to, LepA and LepB. The inclusion of the signal peptidase proteins in the supported lipid bilayer may permit biological molecule generation schemes in which biological molecules are generated with translocation signal sequences to facilitate translocation across the supported lipid bilayer that are subsequently removed to generate pure and complete biological molecules. The supported lipid bilayer may comprise one or more signal peptide hydrolase proteins. The signal peptide hydrolase proteins may be configured to digest the signal peptide that may remain in the membrane after it is cleaved by the signal peptidase. The inclusion of the signal peptide hydrolase may reduce buildup of signal peptides in the membrane an improve longevity of the membrane. The signal peptide proteins may be inserted into the membrane using cell-free expression, mediated insertion (e.g., insertion mediated by SecYEG), or the like.

Subsequently to operation 130, the process 100 may comprise removing an N-terminal translocation signal sequence from the biological molecule. molecule. For example, a signal peptidase can be used to cleave the N-terminal translocation signal sequence from the biological molecule, thus generating the biological molecule. Alternatively, the biological molecule may be generated without an N-terminal translocation signal sequence. The biological molecule may be generated with other additional sequences (e.g., other signaling sequences, secondary domains, etc.). the other additional sequences may be removed from the biological molecule subsequent to the formation of the biological molecule.

In some cases, operation 140 may be performed. In operation 140, the process 100 may comprise removing the biological molecule from the second portion of the chamber. The removing the biological molecule may comprise destruction of the supported lipid bilayer. For example, pressurized gas can be used to force a solution comprising the biological molecule out of the second portion. The removing the biological molecule may comprise a flow of solvent. For example, a flow-cell apparatus can be used to collect the biological molecule from the second portion. The removing may comprise one or more purification operations. Examples of purification operations include, but are not limited to, chromatographic operations (e.g., affinity chromatography, size exclusion chromatography, ion exchange chromatography), extraction operations (e.g., solvent extractions, salt formation reactions, etc.), centrifugation operations (e.g., filter centrifugation, ultracentrifugation, etc.), filtration operations (e.g., paper filtration, tangential flow filtration, ultrafiltration, diafiltration, etc.), lyophilization operations, magnetic separation (e.g., removal of metal nanoparticle tagged reagents/chaperones, etc.) or the like, or any combination thereof. For example, the removing may comprise passing a solution comprising the biological molecule through a filter and a gel chromatography column. The removing may comprise at least about 0, 1, 2, 3, 4, 5, 6, 7, 8, 9, 10, or more purification operations. The removing may comprise at most about 10, 9, 8, 7, 6, 5, 4, 3, 2, 1, or less purification operations. The removing may comprise no purification operations. Subsequent to the removing, reagents separated from the biological molecule may be reused for formation of other biological molecules.

In another aspect, the present disclosure provides a system for generating a biological molecule. The system may comprise a chamber. The chamber may comprise a first portion configured to comprise a plurality of cell-free precursors of the biological molecule. The chamber may comprise a second portion. The chamber may comprise a porous membrane separating the first portion from the second portion. The porous membrane may comprise a lipid bilayer. The lipid bilayer may comprise one or more translocon proteins. The lipid bilayer and the translocon proteins may be as described elsewhere herein. The biological molecule may be a biological molecule as described elsewhere herein. The lipid bilayer may be a supported lipid bilayer.

The porous membrane may comprise a polymer membrane. The polymer membrane may comprise polysulfone, polyethersulfone, polytetrafluoroethylene, polymethylmethacrylate, polyacrylonitrile butadiene styrene, a polyamide, polylactic acid, polybenzimidazole, polycarbonate, polyether sulfone, polyoxymethylene, polyetherether ketone, polyetherimide, polyethylene, polyphenylene oxide, polyphenylene sulfide, polypropylene, polystyrene, polyvinyl chloride, polyvinylidene fluoride, or the like, or any combination thereof. The polymer membrane may be hydrophilic. For example, the porous membrane may comprise hydrophilic polysulfone. The polymer membrane may be hydrophobic. The polymer membrane may be functionalized. For example, a polymer membrane can be treated with ozone to generate surface hydroxy groups on the polymer. The polymer membrane may have a molecular weight cutoff of at least about 1, 5, 10, 15, 20, 25, 30, 35, 40, 45, 50, 55, 60, 65, 70, 75, 80, 85, 90, 95, 100, 110, 120, 130, 140, 150, 175, 200, or more kilodaltons. The polymer membrane may have a molecular weight cutoff of at most about 200, 175, 150, 140, 130, 120, 110, 100, 95, 90, 85, 80, 75, 70, 65, 60, 55, 50, 45, 40, 35, 30, 25, 20, 15, 10, 5, 1, or less kilodaltons. The polymer membrane may have a molecular weight cutoff in a range as defined by any two of the proceeding values. For example, the polymer membrane can have a molecular weight cutoff of about 80 to 110 kilodaltons. The porous membrane may comprise a mesoporous material. Examples of mesoporous materials include, but are not limited to, mesoporous metal oxides (e.g., mesoporous alumina, mesoporous titanium oxide, etc.), mesoporous silica, mesoporous salts (e.g., mesoporous magnesium carbonate, etc.), and mesoporous carbon. The porous membrane may be a treated membrane. Examples of treatments include, but are not limited to, applying one or more other materials to the membrane (e.g., metal plating, polymer coating, etc.), functionalizing the membrane (e.g., applying one or more chemical species to the membrane (e.g., carboxylated polymers, surfactants, passivants, etc.)), or the like, or any combination thereof. The porous membrane may comprise other materials which may comprise pores (e.g., be porous), such as, for example a porous glass substrate, a porous dielectric material substrate, a porous metal substrate, a porous fiber-based substrate (e.g., a paper substrate), or the like, or any combination thereof. The porous membrane may be a positively charged membrane or a membrane treated to have a positive charge (e.g., treated with poly-l-lysine). The lipid bilayer may comprise a negative charge (e.g., be an E. coli polar lipid extract), so a positively charged membrane may aid the formation of the supported lipid bilayer. The porous membrane may be a negatively charged membrane or a membrane treated to have a negative charge.

The supported lipid bilayer may comprise one or more proteins. The one or more proteins may be configured to translocate the biological molecule across the membrane. For example, the biological molecule can pass through a pore in the membrane formed by a protein. The one or more proteins may comprise one or more translocon proteins. For example, the one or more translocon proteins may comprise SecYEG. The one or more proteins may comprise one or more injectosomes. The one or more proteins may comprise one or more hemolysins (e.g., alpha-hemolysin). The one or more proteins may comprise one or more pore forming toxins. The one or more proteins may be one or more proteins as described elsewhere herein.

The plurality of cell-free precursors may not comprise one or more cells. The plurality of cell-free precursors may be generated by lysis and homogenization of one or more cells. For example, a plurality of E. coli cells can be lysed, and the contents of the cells can be used as cell-free precursors. The one or more cell-free precursors may comprise deoxyribonucleic acid (DNA), ribonucleic acid (RNA), one or more amino acids, one or more cofactors (e.g., magnesium, iron, vitamins, minerals, etc.), ribosomes, synthetases, nucleases, or the like, or any combination thereof. The DNA and/or the RNA may encode for the biological molecule. For example, the DNA can encode for the amino acids of a polypeptide. Alternatively, the first portion may comprise one or more cells. The cells may be configured to generate the biological molecule, and the membrane can be used to separate the biological molecule from the cells. For example, the cells can secret the biological molecule into solution in the first portion. In this example, the solution can comprise either a chaperone configured to maintain the biological molecule in an unfolded state (e.g., SecB) and an active transport body (e.g., SecA ATPase, a molecular motor) to translate the biological molecule across the lipid bilayer or vesicles (e.g., fusogenic vesicles) configured to shuttle the biological molecule to the lipid bilayer, fuse with the lipid bilayer, and thus transport the biological molecule to the second portion. In this example, an injectosome may be used to transport the biological molecule through the lipid bilayer. The plurality of cell-free precursors may not comprise inverted membrane vesicles. The inverted membrane vesicles may be byproducts of a cell lysis. For example, an inner membrane of a cell can form an inverted membrane vesicle upon cell lysis. The inverted membrane vesicles may comprise proteins native to the cell that was lysed. For example, an inverted membrane vesicle may comprise native SecYEG proteins. The elements native to the cell may compete with proteins in the supported lipid bilayer. For example, native Sec YEG from a lysed cell can compete with SecYEG present in a supported lipid bilayer. In this example, there can be more native SecYEG than SecYEG in the bilayer, and the native SecYEG can outcompete the SecYEG in the bilayer, which can in turn decrease yields across the bilayer.

The inverted membrane vesicles may be removed from the plurality of cell-free precursors. The cell-free precursors may be as described elsewhere herein. For example, the cell-free precursors can comprise nucleic acids, amino acids, chaperones, or the like, or any combination thereof. The proteins in the inverted membrane vesicles may be selectively inactivated. For example, the cells that are lysed to form the cell-free precursors can comprise an engineered native SecYEG protein can be configured to function normally during cell growth and development but be inactivated after lysis. In this example, the functionality of the native SecYEG can be destroyed after lysis in order to remove a competing pathway with SecYEG present in the supported lipid bilayer. Instead of using a plurality of cell-free precursors from a lysed cell, a purified and reconstituted system may be used. For example, the plurality of cell-free precursors can each be separately purified and subsequently mixed together to form a purified solution of the plurality of cell-free precursors. In this example, additional precursors can be added to the solution depending on the use of the solution (e.g., adding proteins to enable translocation of the supported lipid bilayer).

The second portion may comprise a different environment from the first portion. The different environment may be a different temperature, solvent system (e.g., polarity, solvent mixture, etc.), ionic strength, presence or absence of other molecules (e.g., cofactors, binding substrates, etc.), presence of absence of chaperone molecules, presence or absence of post-translational modification enzymes, or the like, or any combination thereof. For example, the second portion may be held at a lower ionic strength than the first portion. In this example, electrostatic screening may be lower in the second portion, thus permitting increased interaction between different portions of the biological molecule.

In another aspect, the present disclosure provides a method for generating a cell-free synthesis chamber. The method may comprise providing a chamber comprising a first portion and a second portion. The first portion and the second portion may be separated by a porous membrane. A solution comprising a plurality of proteoliposomes may be applied to the porous membrane. The plurality of proteoliposomes may comprise a lipid bilayer and one or more translocon proteins. The plurality of proteoliposomes may be reacted with the porous membrane. The reacting may comprise dissociation of the plurality of proteoliposomes to form a supported lipid bilayer on the porous membrane. The supported lipid bilayer may comprise the one or more translocon proteins.

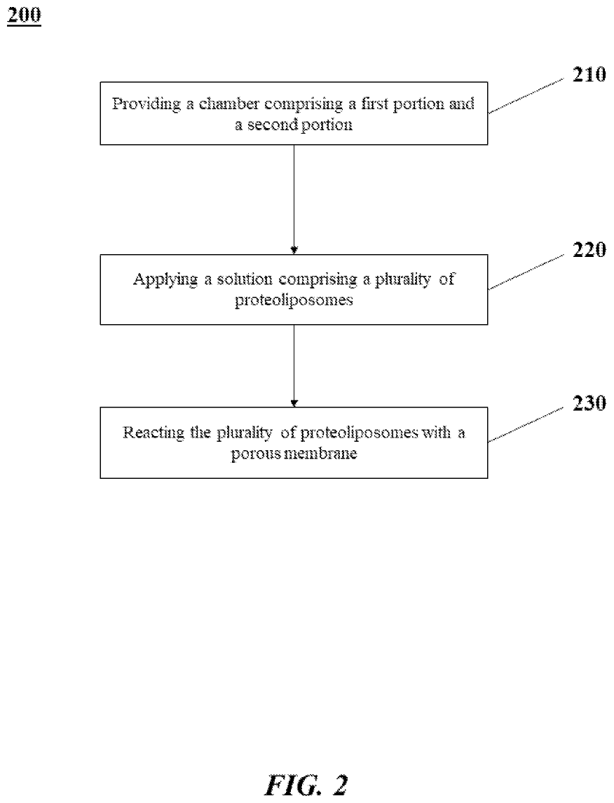

FIG. 2 is an example of a flowchart for a process 200 for generating a cell-free synthesis chamber. In an operation 210, the process 200 may comprise providing a chamber comprising a first portion and a second portion. The first portion may be separated from the second portion by a porous membrane. The chamber may be a chamber as described elsewhere herein. For example, the chamber may be a flow chamber.

In another operation 220, the process 200 may comprise applying a solution comprising a plurality of proteoliposomes. The plurality of the proteoliposomes may comprise a lipid bilayer and one or more translocon proteins. In some cases, the plurality of proteoliposomes may not comprise one or more translocon proteins. For example, the proteoliposomes can be liposomes. In this example, the liposomes can be used to generate a supported lipid bilayer, and subsequent to the forming of the supported lipid bilayer, one or more translocon proteins may be added to the supported lipid bilayer. Other ways of forming supported lipid bilayers may be used as well, such as, for example, lipid stacking followed by plasma etching.

The solution may comprise a plurality of liposomes without the one or more translocon proteins. The liposomes without the one or more translocon proteins may be of a same composition as the plurality of proteoliposomes. For example, the liposomes and the proteoliposomes can both comprise POPC. The concentration of translocon proteins may be tuned to a predetermined value by adjusting the ratio of the liposomes to the proteoliposomes. For example, a lipid bilayer with dilute translocon proteins can be formed by generating a solution with more liposomes than proteoliposomes and applying the solution to the porous membrane.

The proteoliposomes and/or the liposomes may be substantially homogeneous in size. The proteoliposomes and/or the liposomes may have a size distribution of at least about 1%, 2%, 3%, 4%, 5%, 6%, 7%, 8%, 9%, 10%, or more. The proteoliposomes and/or the liposomes may have a size distribution of at most about 10%, 9%, 8%, 7%, 6%, 5%, 4%, 3%, 2%, 1%, or less. The proteoliposomes and/or the liposomes may be generated by extrusion through a pore. The proteoliposomes and/or the liposomes may be generated by rehydration of lipids in an aqueous solution. The proteoliposomes and/or the liposomes may be generated by sonication. The proteoliposomes and/or the liposomes may be formed by other processes such as, for example, those described in “Novel methods for liposome preparation” by Patil et al., Chemistry and Physics of Lipids Volume 177, January 2014, Pages 8-18 DOI number 10.1016/j.chemphyslip.2013.10.011, which is incorporated by reference in its entirety. The proteoliposomes and/or the liposomes may have a size of at least about 10, 50, 100, 250, 500, 1,000, or more nanometers. The proteoliposomes and/or the liposomes may have a size of at most about 1,000, 500, 250, 100, 50, 10, or less nanometers.

The proteoliposomes may be generated by incubation of liposomes with cell-free precursors of the translocon proteins. For example, the translocon proteins can be generated in a cell-free reaction using RNA and/or DNA that encodes for the translocon proteins and cell-free ribosomes and the liposomes can be introduced into the cell-free reaction mixture and incubated until the translocon proteins are incorporated into the liposomes. The proteoliposomes may be generated by rehydrating dried translocon proteins with a solution comprising liposomes. For example, a solution comprising translocon proteins can be lyophilized and subsequently rehydrated in a solution comprising proteoliposomes. The proteoliposomes may be generated by a detergent exchange method. For example, proteins solubilized in a detergent can be added to a solution comprising micelles, and the proteins can be exchanged from the detergent to incorporate into the micelles. In another example, proteins solubilized in a detergent can be added to a solution comprising unilamellar vesicles, and the proteins can be exchanged from the detergent to incorporate into the unilamellar vesicles. The proteoliposomes may be generated by mixing a translocon protein solution with a liposome solution. For example, the translocons can integrate into the liposomes in solution.

In another operation 230, the process 200 may comprise reacting the plurality of proteoliposomes with the porous membrane. The reacting may comprise dissociation of the plurality of proteoliposomes to form a supported lipid bilayer on the porous membrane. The supported lipid bilayer may comprise the one or more translocon proteins. In some cases, the supported lipid bilayer may be generated on the porous membrane separate from a chamber. For example, the supported lipid bilayer may be generated on a porous membrane in a reaction vessel configured for formation of supported lipid bilayers. In this example, the porous membrane comprising the supported lipid bilayer can be removed from the reaction vessel and placed within a chamber as described elsewhere herein. In some cases, the supported lipid bilayer may be formed without any translocon proteins. The translocon proteins may be added to the supported lipid bilayer subsequent to the formation of the supported lipid bilayer. For example, a supported lipid bilayer can be formed on the porous membrane and a solution comprising the translocon proteins can be introduced to the supported lipid bilayer and the translocon proteins can integrate into the supported lipid bilayer. The supported lipid bilayer may be generated from inverted membrane vesicles. The inverted membrane vesicles may be derived from one or more cells. For example, E. coli can be configured to generate translocon proteins, either natively or with genetic engineering, and the cells of the E. coli can be transformed into inverted membrane vesicles that may subsequently be reacted to form a supported lipid bilayer comprising translocon proteins. The process of generating a supported lipid bilayer from inverted membrane vesicles may be similar to generating a supported lipid bilayer from proteoliposomes. For example, the inverted membrane vesicles may be reacted with a porous substrate to form a supported lipid bilayer on the porous substrate. The membrane may be a lipid bilayer. The lipid bilayer may be supported by one or more substrates. In some examples, the lipid bilayer is supported by substrates (e.g., sandwiched between two substrates). The membrane may be a solid-state membrane, such as, for example, a dielectric. The solid-state membrane may be formed of a silicon oxide or a silicon nitride, for example.

Instead of providing proteoliposomes to a lipid bilayer to form a lipid bilayer comprising translocon proteins, the lipid bilayer comprising translocon proteins can be formed by incubating a lipid bilayer in a solution comprising precursors for the translocon proteins. The precursors may comprise a plurality of cell-free precursors as described elsewhere herein. The precursors may comprise a nucleic acid template for the translocon proteins. For example, the precursors can comprise a DNA template for a SecYEG complex. The cell-free precursors can be configured to synthesize the translocon protein, which can then incorporate directly into the lipid bilayer.

Such direct incorporation of freshly synthesized translocon proteins can produce a membrane with a higher translocation efficiency than one produced by proteoliposome rupture. For example, a directly incorporated membrane can have all of the translocon proteins oriented in the correct direction. Direct incorporation made membranes may also be lower cost than proteoliposome rupture-based membranes. For example, the rupture-based membranes can be formed from purified proteoliposomes while direct insertion translocon proteins may not be purified. By using directly incorporated proteins, a single operation can be executed to form the supported lipid bilayer with the integrated proteins. For example, a cell-free precursor mixture along with DNA associated with the integrated proteins can be provided adjacent to a supported lipid bilayer. In this example, the integration of the translocon protein (e.g., SecYEG), the incorporation of a cofactor to the translocon protein (e.g., LepB into the SecYEG), and expression of tethered chaperones as described elsewhere herein can all be completed in a same operation. In this example, the timing of the introduction of the various DNA's can be adjusted to provide for optimal efficiency (e.g., the DNA corresponding to SecYEG can be introduced prior to the DNA corresponding with LepB). The biological molecule may be expressed at a same time as the translocon proteins. For example, the cell-free solution can comprise components configured to form the biological molecule, which can in turn translocate through the freshly produced translocon proteins. An additional benefit of introducing translocon proteins directly may be a simpler transport procedure for the supported lipid bilayer. For example, a bilayer that does not comprise proteins can be more robust and lower maintenance in shipping than a bilayer that comprises proteins.

In another aspect, the present disclosure provides a method for generating a polypeptide. The method may comprise using a cell-free solution comprising a deoxyribonucleic acid molecule encoding the polypeptide to generate a ribonucleic acid molecule. The ribonucleic acid molecule may be used to generate the polypeptide. The polypeptide may be directed through a pore disposed in a membrane.

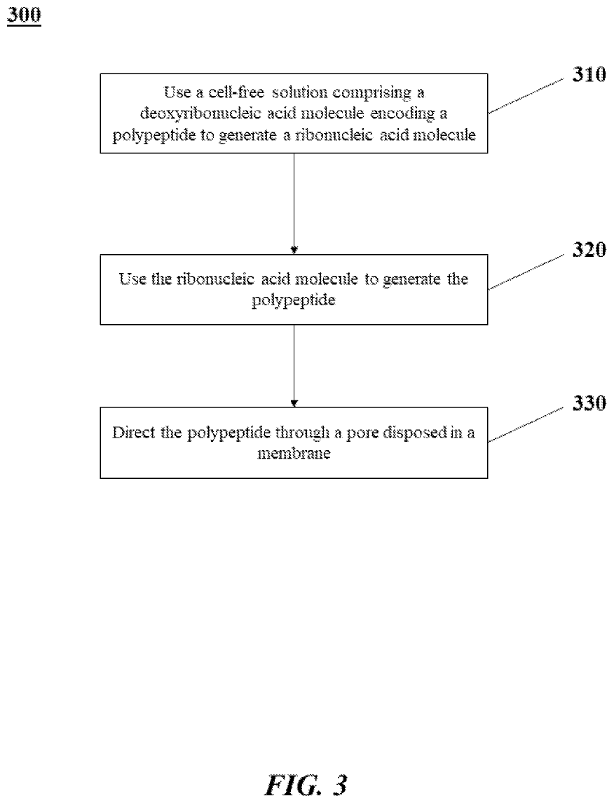

FIG. 3 is an example flowchart for a process 300 for generating a polypeptide. In an operation 310, the process 300 may comprise using a cell-free solution comprising a deoxyribonucleic acid molecule encoding a polypeptide to generate a ribonucleic acid molecule. Alternatively, the ribonucleic acid molecule may be introduced to the cell-free solution already generated. For example, a ribonucleic acid encoding a protein can be introduced to a cell-free solution that does not comprise DNA.

In another operation 320, the process 300 may comprise using the ribonucleic acid molecule to generate the polypeptide. The generation may comprise use of one or more cellular bodies (e.g., ribosomes, peptidases, etc.).

In another operation 330, the process 300 may comprise directing the polypeptide through a pore disposed in a membrane. The pore may comprise a protein. The protein may comprise a translocon protein. The membrane may be a supported lipid bilayer. The membrane may be another membrane as described elsewhere herein. The pore and the membrane may be as described elsewhere herein. The polypeptide may comprise a non-native N-terminal signal sequence. For example, the RNA may encode for a non-wildtype polypeptide that has been configured to comprise a terminal signal sequence. The terminal signal sequence may be configured to be removed by a signal peptidase protein.

The membrane may comprise a supported lipid bilayer. The membrane may not be a part of a micelle. For example, the membrane may not be a membrane free in solution. The membrane may be planar. For example, the membrane may be a planar supported lipid bilayer on a support. The membrane may be substantially planer. For example, the membrane can be applied to a rough support. The pore may comprise one or more translocon proteins. The one or more translocon proteins may be as described elsewhere herein. The membrane may comprise one or more signal peptidase proteins and/or one or more other proteins as described elsewhere herein. The membrane may be rolled into a hollow fiber configuration (e.g., rolled into a tube).

Subsequent to operation 330, the polypeptide may be present at a purity of at least about 20%, 30%, 40%, 50%, 55%, 60%, 65%, 70%, 75%, 80%, 85%, 90%, 95%, 96%, 97%, 98%, 99%, or more. Subsequent to operation 330, the polypeptide may be present at a purity of at most about 99%, 98%, 97%, 96%, 95%, 90%, 85%, 80%, 75%, 70%, 65%, 60%, 55%, 50%, 40%, 30%, 20%, or less. The polypeptide may be present at one of the aforementioned purities without additional purification operations. For example, subsequent to moving through the pore, the polypeptide can be at a purity of at least about 60%. The polypeptide may be used without further purification subsequent to operation 330. The purity may be a purity of the molecular weight of the biological molecule (e.g., a size distribution of the completed biological molecule), a molarity of the biological molecule, a ratio of the biological molecule to other molecules in solution, a ratio of the biological molecule to other biological molecules in solution, or the like, or any combination thereof. The purity may be a purity in a second portion of a chamber as described elsewhere herein. The purity may be a purity of biological molecules in a membrane as described elsewhere herein.

Operations 310-330 may be performed within a time period of at least about 30 seconds, 1 minute (m), 5 m, 10 m, 15 m, 30 m, 1 hour (h), 2 h, 3 h, 4 h, 5 h, 6 h, 7 h, 8 h, 9 h, 10 h, 12 h, 18 h, 24 h, 48 h, 72 h, 96 h, or more. Operations 310-330 may be performed within a time period of at most about 96 h, 72 h, 48 h, 24 h, 18 h, 12 h, 10 h, 9 h, 8 h, 7 h, 6 h, 5 h, 4 h, 3 h, 2 h, 1 h, 30 m, 15 m, 10 m, 5 m, 1 m, 30 s, or less.

In another aspect, the present disclosure provides a system for generating a biological molecule. The system may comprise a chamber. The chamber may comprise a first portion configured to contain a plurality of cell-free precursors of the biological molecule. The chamber may comprise a second portion. The chamber may comprise a porous membrane separating the first portion from the second portion. The porous membrane may comprise a supported lipid bilayer. The supported lipid bilayer may comprise one or more signal peptidase proteins.

The one or more signal peptidase proteins may comprise one or more signal peptidase proteins as described elsewhere herein. For example, the one or more signal peptidase proteins may comprise LepB. The supported lipid bilayer may comprise one or more translocon proteins as described elsewhere herein. For example, the supported lipid bilayer may be configured to permit translocation of the biological molecule through the porous membrane through the one or more translocon proteins.

FIGS. 4A-4D are examples of a method for generating a flow cell chamber. A flow chamber 601 may comprise two or more flow ports 602. The chamber 601 may comprise at least about 2, 3, 4, 5, 6, 7, 8, 9, 10, or more flow ports. The chamber 601 may comprise at most about 10, 9, 8, 7, 6, 5, 4, 3, or fewer flow ports. For example, the chamber can comprise two flow ports on one side of a membrane and two flow ports on the other side of the membrane. The flow ports may be in fluidic communication with one or more reservoirs (e.g., reagent reservoirs, wash reservoirs, etc.), waste handling (e.g., waste disposal), characterization instrumentation (e.g., chromatography, mass spectrometry, nuclear magnetic resonance, optical, etc.), lab-on-a-chip functionalities (e.g., those described elsewhere herein), other chambers configured to generate other biomolecules (e.g., the output of a first chamber is a portion of the cell-free reaction mixture of the second chamber), or the like, or any combination thereof. The flow ports may be on a same side of the chamber. For example, all of the flow ports can be on the side of the chamber in order to permit easy insertion and removal from a larger system.

The chamber may comprise a first portion 604 and a second portion 605 separated by a membrane 603. The membrane may be a membrane as described elsewhere herein. For example, the membrane may comprise a mesoporous membrane. In the process of generating a supported lipid bilayer on the membrane, a plurality of translocon-containing proteoliposomes can be flowed into the first and/or second portions of the chamber via the flow ports 602. In the example of FIG. 4B, the solution 606 can be flowed into the first portion. The solution may be described as elsewhere herein. For example, the solution may comprise a plurality of both proteoliposomes and liposomes. The solution may be reacted with the membrane as described elsewhere herein. For example, the solution can be incubated with the membrane and reacted to form a supported lipid bilayer on the membrane. The supported lipid bilayer may comprise one or more translocon and or signal peptidase proteins as described elsewhere herein.

After the reaction to form a supported lipid bilayer 607 comprising the translocon and/or signal peptidase proteins, a cell-free reaction mixture 608 may be introduced into the chamber via one or more flow ports as shown in FIG. 4C. The cell-free reaction mixture may be flowed into the first or the second portion. The cell-free reaction mixture may be as described elsewhere herein. The chamber may be configured to hold the cell-free reaction mixture under conditions sufficient for the formation of one or more biological molecules 609 as described elsewhere herein. The one or more biological molecules may translocate through the membrane to the other portion of the chamber. Once in the other portion, the biological molecules may remain in the other portion when not subjected to a flow. Alternatively, if a flow is present in the other portion, the biological molecules may be flowed out of the chamber through one of the flow ports.

FIG. 4D is an example of a wash operation subsequent to the formation of the one or more biological molecules. A wash 610 may be flowed into the chamber to wash the cell-free reaction mixture and/or the biological molecules out of the chamber. The was operation may comprise flow of fluid to one portion of the chamber but not the other portion of the chamber. For example, a pressurized wash can be applied to the first portion, and the biological molecule can be driven to the second portion by the pressure. In another example, the second portion can be washed to remove the biological molecule while the first portion is not washed. Subsequent to the wash, the chamber may be reused for the generation of the same or different biological molecules. For example, the chamber can be treated with a DNase and/or an RNase to remove remaining reactants. In this example, a DNase and/or RNase inhibitor can be introduced prior to reintroduction of the cell-free precursors. In some cases, the wash may be performed in a continuous manner. For example, the wash can be flowed through the chamber constantly to affect a continuous removal of the biological molecule from the chamber. This continuous removal can improve the yield of the reaction. The wash may be performed in a semi-continuous (e.g., batch) method. For example, the wash can be performed for 30 minutes every 3 hours. Removing the biological molecule from the chamber can induce a concentration gradient. The concentration gradient may result in the biological molecule being drawn down from the membrane, which may improve the yield of the biological molecule.

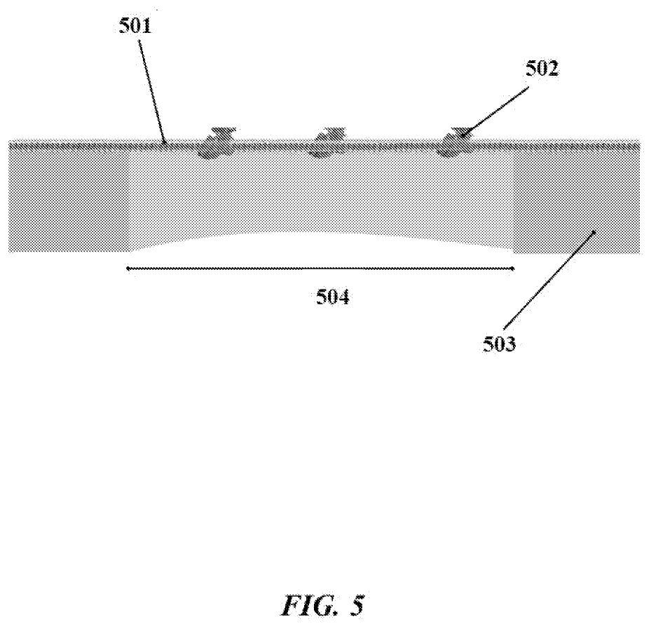

FIG. 5 is an example of a supported lipid bilayer 501 comprising proteins 502. Although the supported lipid bilayer 501, as illustrated, comprises three proteins 502, the supported lipid bilayer 501 may comprise at least 1, 2, 3, 4, 5, 6, 7, 8, 9, 10, 20, 30, 40, 50, 100, 200, 300, 400, 500, 1000 or more proteins 502. The supported lipid bilayer may be a supported lipid bilayer as described elsewhere herein. For example, the supported lipid bilayer may comprise 1-palmitoyl-2-oleoyl-sn-glycero-3-phosphocholine. The proteins may comprise translocon proteins as described elsewhere herein, signal peptidase proteins as described elsewhere herein, or a combination thereof. The positioning of the proteins above the pore may permit the transit of biological molecules through the bilayer 501 through pores in the translocon proteins.

The supported lipid bilayer may be supported on membrane 503. The membrane may be a membrane as described elsewhere herein. For example, the membrane can comprise mesoporous alumina, mesoporous silica, or mesoporous polysulfone. The membrane may comprise one or more pores 504. The pore may have a size of at least about 10 nanometers (nm), 25 nm, 50 nm, 75 nm, 100 nm, 150 nm, 200 nm, 250 nm, 500 nm, 750 nm, 1,000 nm, or more. The pore may have a size of at most about 1,000 nm, 750 nm, 500 nm, 250 nm, 200 nm, 150 nm, 100 nm, 75 nm, 50 nm, 25 nm, 10 nm, or less.

The membrane 503 may comprise additional structures such as, for example, electrodes, electrical leads, temperature sensors, proteins, cellular bodies, organelles, or the like, or any combination thereof. For example, protein generating organelles can be tethered to the membrane adjacent to the pores to permit translocation of the protein upon generation by the organelle. In another example, the membrane can comprise electrodes configured to generate an electric field to direct flow of biological molecules through the pore.

In some cases, a folding control scheme (e.g., controlled folding of a biological molecule) can be implemented in a proteoliposome based biological molecule production method. In some cases, such a scheme can be configured to produce the biological molecule without the use of a supported lipid bilayer, which can provide a simplified production scheme. The proteoliposome based biological molecule production method can provide benefits such as, for example, improved folding control, rapid synthesis of the biological molecule, and tag-free synthesis of the biological molecule.

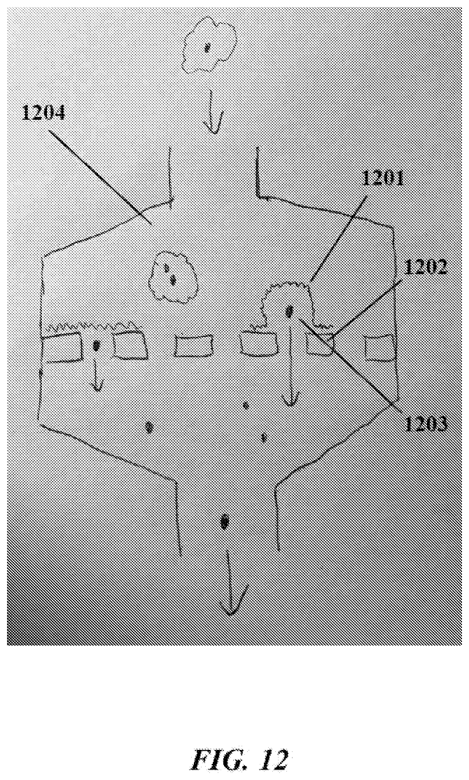

The proteoliposome based production method can comprise performing an expression and translocation reaction. The expression and translocation reaction may be a one-pot (e.g., in a single container) reaction. For example, the expression and translocation of the biological molecule can occur in a single container. The one-pot reaction may comprise the proteoliposomes or liposomes (e.g., the translocon protein complexes and/or chaperones can be expressed and introduced into the liposomes subsequent to addition to the container), transcription and/or translation reagents, translocation components (e.g., SecA, SecB, SRP, Ffh, Ffs, FtsY, or the like, or any combination thereof), one or more template nucleic acids (e.g., DNA) corresponding to the product, one or more template nucleic acids corresponding to, for example, translocon proteins, chaperones, or the like, or the like, or any combination thereof. The proteoliposomes may comprise one or more translocon proteins (e.g., SecYEG), one or more signal peptidase proteins (e.g., LepB), one or more chaperones (e.g., chaperones comprised within the proteoliposome, chaperones tethered to the proteoliposome, a combination thereof, et.), a buffer comprised within the proteoliposome (e.g., a buffer configured to provide improved quality of the biological molecule), or the like, or any combination thereof. The proteoliposomes may be subjected to conditions sufficient for the production of the biological molecules as described elsewhere herein. The resultant proteoliposomes comprising the biological molecules may be separated from the reaction mixture. Examples of separation techniques include, but are not limited to, chromatography (e.g., affinity chromatography, size exclusion chromatography, etc.), ultracentrifugation (e.g., sucrose gradient ultracentrifugation, etc.), or the like, or any combination thereof. The biological molecules may be removed from the proteoliposomes by, for example, sonication, vortexing, detergent addition, electroportation, or the like, or any combination thereof. The resultant proteoliposome residue may be removed from the biological molecules by, for example, chromatography or centrifugation as described elsewhere herein, use of a porous membrane (e.g., with pores configured to permit flow of the biological molecule through the porous membrane while excluding flow of the proteoliposome residue), or the like, or any combination thereof. In some cases, the biological molecule may be removed from the proteoliposome via rupturing the proteoliposome onto a porous substrate (e.g., similar to the production of a supported lipid bilayer). The biological molecule can diffuse or be flowed through the pores of the porous substrate upon release from the proteoliposome, and the biological molecule can be collected from the other side of the porous substrate. FIG. 12 shows an example of a proteoliposome 1201 being ruptured on a porous substrate 1202 to release the biological molecule 1203 in a reactor 1204. By rupturing the proteoliposome on a porous substrate, the biological molecule can be collected without a residue from the proteoliposome, and thus as a more pure product, without the use of a purification process (e.g., chromatography, centrifugation, etc.).

In some cases, a biomolecule (e.g., chaperone, enzyme, or the like) may be immobilized to the proteoliposome or to a supported lipid bilayer as described elsewhere herein. The immobilization may comprise use of a biotin/streptavidin binding pair. For example, streptavidin can be immobilized to a supported lipid bilayer or proteoliposome while the biomolecule is functionalized with biotin, or vice versa. The streptavidin can be introduced to the lipid bilayer as a purified product (e.g., prepared prior or prepared and passed through a lipid bilayer, etc.) or expressed from a cell-free reaction and introduced to the lipid bilayer. The streptavidin can comprise a non-cleavable signal sequence and an optional linker to tether the streptavidin to the lipid bilayer. The biotin can be included in the lipid bilayer by way of biotinylated lipids. In some cases, the biomolecule can be expressed as a streptavidin fusion. The biomolecule streptavidin fusion may be expressed as a cell-free reaction or introduced as a purified fusion.

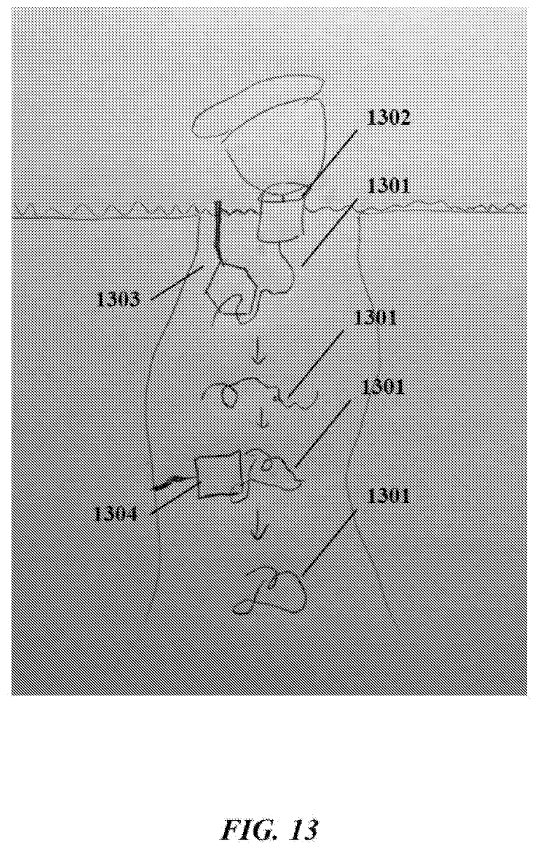

In some cases, the termini of the biomolecule may be important, thereby limiting the use of a streptavidin fusion or other methods of tethering or anchoring to the lipid bilayer. This may be addressed with a linker between the biomolecule and a signal sequence. In some cases, the biomolecule can be biotinylated and the lipid bilayer can comprise streptavidin. In some cases, unnatural amino acids can be incorporated as a side chain to the biomolecule and be configured to bind to a target group (e.g., via click chemistry, etc.). The unnatural amino acids may be placed to avoid functional interference with the biomolecule's function. The target group may be immobilized to the lipid bilayer (e.g., lipids can be functionalized with the target group). The target group may comprise a linker (e.g., configured to avoid steric hindrance between the biomolecule and the lipid bilayer). A density of functionalized lipids may be controlled to control a density of biomolecules immobilized thereto. Different biomolecules may be immobilized at different times. For example, a first biomolecule acting as a early-stage chaperone (e.g., DnaKJE complex) can be immobilized prior to a second biomolecule (e.g., GroEL/GroES) acting as a late-stage chaperone. Different chaperones can be introduced to the biological molecule at different times by use of the diffusion of the biomolecule from the lipid bilayer through a porous support into a second reaction chamber. For example, a biological molecule produced in a first reaction chamber and diffused through a supported lipid bilayer can enter a pore of the porous support before the second reaction chamber close to the supported lipid bilayer. The time taken for the biological molecule to diffuse through the lipid bilayer and pores can be matched with the time the biological molecule is to interact with various chaperones (e.g., the time in different folding regimes). For example, an early folding chaperone can be tethered to the lipid bilayer to interact with the biological molecule upon translocation through the lipid bilayer, while a later folding chaperone can be immobilized to the interior of a pore to interact with the biological molecule subsequent to the early folding chaperone. In this example, another chaperone can be included in the second chamber to interact with the biological molecule subsequent to the later folding chaperone. FIG. 13 shows an example of a biological molecule 1301 traversing a pore in supported lipid bilayer 1302. Upon translocation, the biological molecule can interact with a first chaperone 1303 bound to the lipid bilayer. The first chaperone can aid a first folding of the biological molecule to put the biological molecule in a first folded state. A second chaperone 1304 can be immobilized to the interior of a pore of the porous membrane the supported lipid bilayer is formed on, and can aid a second folding of the biological molecule into a second folded state. Additional chaperones may be immobilized to the porous support, lipid bilayer, or be present in a solution in the second reactor chamber to affect additional foldings of the biological molecule.

In some cases, a biomolecule can be modified with an immobilization reagent and not immobilized to the proteoliposome or supported lipid bilayer as described elsewhere herein. The modified biomolecule may be introduced to a complimentary immobilizing reagent subsequent to use of the biomolecule to remove the biomolecule from solution. For example, a biotinylated biomolecule can be purified from solution by introduction of a surface comprising streptavidin.

A proteoliposome may be generated by shedding the proteoliposome from an engineered cell. For example, E. coli can be engineered to shed SecYEG rich viral particles after overexpressing SecYEG. The proteoliposomes can be removed from the engineered cells by, for example, size exclusion chromatography. Generating proteoliposomes in this way may avoid a lysis operation on the cells, thereby reducing impurities generated by cell lysis.

In some cases, the supported lipid bilayer described elsewhere herein may be replaced with a dialysis plate. For example, proteoliposomes comprising translocon proteins can be introduced to and fused to a dialysis membrane, which can be used in the methods and systems described herein in place of a supported lipid bilayer. For example, a cell-free precursor solution can be introduced to the dialysis chamber, and product biological molecules can be translocated through the translocon proteins. The biological molecule can then be collected from the other side of the dialysis plate (e.g., from a well, tube, plate, etc.).

### Computer Systems

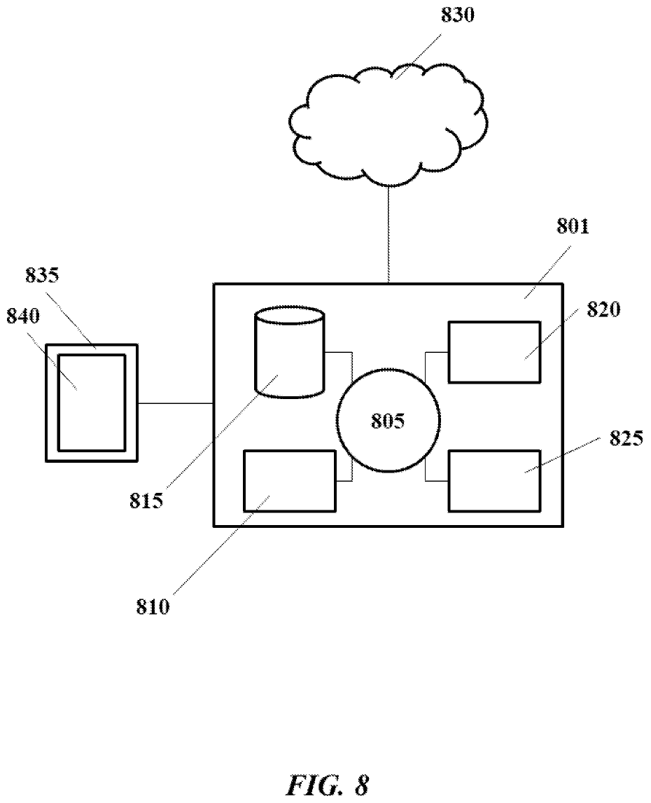

The present disclosure provides computer systems that are programmed to implement methods of the disclosure. FIG. 8 shows a computer system 801 that is programmed or otherwise configured to perform methods and regulate systems of the present disclosure. The computer system 801 can regulate various aspects of the present disclosure, such as, for example, methods of generating biological molecules or generating cell-free synthesis chambers. For example, a computer system can be configured to control the conditions for the formation of a biological molecule within the chamber. In another example, a computer system can regulate the conditions of the forming of a cell-free synthesis chamber. The computer system 801 can be an electronic device of a user or a computer system that is remotely located with respect to the electronic device. The electronic device can be a mobile electronic device.

The computer system 801 includes a central processing unit (CPU, also “processor” and “computer processor” herein) 805, which can be a single core or multi core processor, or a plurality of processors for parallel processing. The computer system 801 also includes memory or memory location 810 (e.g., random-access memory, read-only memory, flash memory), electronic storage unit 815 (e.g., hard disk), communication interface 820 (e.g., network adapter) for communicating with one or more other systems, and peripheral devices 825, such as cache, other memory, data storage and/or electronic display adapters. The memory 810, storage unit 815, interface 820 and peripheral devices 825 are in communication with the CPU 805 through a communication bus (solid lines), such as a motherboard. The storage unit 815 can be a data storage unit (or data repository) for storing data. The computer system 801 can be operatively coupled to a computer network (“network”) 830 with the aid of the communication interface 820. The network 830 can be the Internet, an internet and/or extranet, or an intranet and/or extranet that is in communication with the Internet. The network 830 in some cases is a telecommunication and/or data network. The network 830 can include one or more computer servers, which can enable distributed computing, such as cloud computing. The network 830, in some cases with the aid of the computer system 801, can implement a peer-to-peer network, which may enable devices coupled to the computer system 801 to behave as a client or a server.

The CPU 805 can execute a sequence of machine-readable instructions, which can be embodied in a program or software. The instructions may be stored in a memory location, such as the memory 810. The instructions can be directed to the CPU 805, which can subsequently program or otherwise configure the CPU 805 to implement methods of the present disclosure. Examples of operations performed by the CPU 805 can include fetch, decode, execute, and writeback.

The CPU 805 can be part of a circuit, such as an integrated circuit. One or more other components of the system 801 can be included in the circuit. In some cases, the circuit is an application specific integrated circuit (ASIC).

The storage unit 815 can store files, such as drivers, libraries, and saved programs. The storage unit 815 can store user data, e.g., user preferences and user programs. The computer system 801 in some cases can include one or more additional data storage units that are external to the computer system 801, such as located on a remote server that is in communication with the computer system 801 through an intranet or the Internet.

The computer system 801 can communicate with one or more remote computer systems through the network 830. For instance, the computer system 801 can communicate with a remote computer system of a user. Examples of remote computer systems include personal computers (e.g., portable PC), slate or tablet PC's (e.g., Apple® iPad, Samsung® Galaxy Tab), telephones, Smart phones (e.g., Apple® iPhone, Android-enabled device, Blackberry®), or personal digital assistants. The user can access the computer system 801 via the network 830.

Methods as described herein can be implemented by way of machine (e.g., computer processor) executable code stored on an electronic storage location of the computer system 801, such as, for example, on the memory 810 or electronic storage unit 815. The machine executable or machine-readable code can be provided in the form of software. During use, the code can be executed by the processor 805. In some cases, the code can be retrieved from the storage unit 815 and stored on the memory 810 for ready access by the processor 805. In some situations, the electronic storage unit 815 can be precluded, and machine-executable instructions are stored on memory 810.

The code can be pre-compiled and configured for use with a machine having a processer adapted to execute the code, or can be compiled during runtime. The code can be supplied in a programming language that can be selected to enable the code to execute in a pre-compiled or as-compiled fashion.

Aspects of the systems and methods provided herein, such as the computer system 801, can be embodied in programming. Various aspects of the technology may be thought of as “products” or “articles of manufacture” typically in the form of machine (or processor) executable code and/or associated data that is carried on or embodied in a type of machine readable medium. Machine-executable code can be stored on an electronic storage unit, such as memory (e.g., read-only memory, random-access memory, flash memory) or a hard disk. “Storage” type media can include any or all of the tangible memory of the computers, processors or the like, or associated modules thereof, such as various semiconductor memories, tape drives, disk drives and the like, which may provide non-transitory storage at any time for the software programming. All or portions of the software may at times be communicated through the Internet or various other telecommunication networks. Such communications, for example, may enable loading of the software from one computer or processor into another, for example, from a management server or host computer into the computer platform of an application server. Thus, another type of media that may bear the software elements includes optical, electrical, and electromagnetic waves, such as used across physical interfaces between local devices, through wired and optical landline networks and over various air-links. The physical elements that carry such waves, such as wired or wireless links, optical links, or the like, also may be considered as media bearing the software. As used herein, unless restricted to non-transitory, tangible “storage” media, terms such as computer or machine “readable medium” refer to any medium that participates in providing instructions to a processor for execution.

Hence, a machine readable medium, such as computer-executable code, may take many forms, including but not limited to, a tangible storage medium, a carrier wave medium or physical transmission medium. Non-volatile storage media include, for example, optical or magnetic disks, such as any of the storage devices in any computer(s) or the like, such as may be used to implement the databases, etc. shown in the drawings. Volatile storage media include dynamic memory, such as main memory of such a computer platform. Tangible transmission media include coaxial cables; copper wire and fiber optics, including the wires that comprise a bus within a computer system. Carrier-wave transmission media may take the form of electric or electromagnetic signals, or acoustic or light waves such as those generated during radio frequency (RF) and infrared (IR) data communications. Common forms of computer-readable media therefore include for example: a floppy disk, a flexible disk, hard disk, magnetic tape, any other magnetic medium, a CD-ROM, DVD or DVD-ROM, any other optical medium, punch cards paper tape, any other physical storage medium with patterns of holes, a RAM, a ROM, a PROM and EPROM, a FLASH-EPROM, any other memory chip or cartridge, a carrier wave transporting data or instructions, cables or links transporting such a carrier wave, or any other medium from which a computer may read programming code and/or data. Many of these forms of computer readable media may be involved in carrying one or more sequences of one or more instructions to a processor for execution.

The computer system 801 can include or be in communication with an electronic display 835 that comprises a user interface (UI) 840 for providing, for example, a control panel for inputting predetermined properties of a biological molecule. Examples of UI's include, without limitation, a graphical user interface (GUI) and web-based user interface.

Methods and systems of the present disclosure can be implemented by way of one or more algorithms. An algorithm can be implemented by way of software upon execution by the central processing unit 805. The algorithm can, for example, cycle a cell-free reaction chamber to generate a biological molecule.

### EXAMPLES

The following examples are illustrative of certain systems and methods described herein and are not intended to be limiting.

### Example 1—Preparation of a Chamber Comprising a Supported Lipid Bilayer

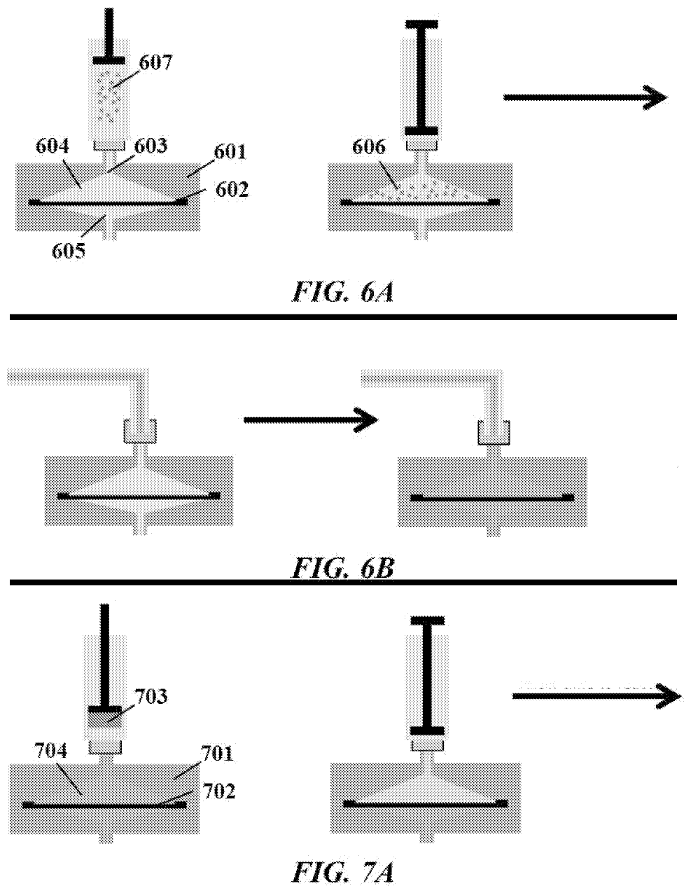

FIGS. 6A and 6B are examples of a process for generating a chamber 601 comprising a membrane 602 and using the chamber to generate a biological molecule. The chamber may comprise an inlet 603. The inlet may be configured to connect to a vessel 607 (e.g., a syringe, a tube, etc.). The vessel may be configured to introduce a plurality of proteoliposomes to the first portion 604 of the chamber 601.

The proteoliposomes may be formed by evaporation of chloroform solvent from POPC lipids using nitrogen gas flow followed by application of vacuum. The dry POPC can be rehydrated in an aqueous buffer and extruded through 100 nm pores to generate liposomes. The liposomes can then be incubated with a cell-free transcription/translation solution with DNA encoding for SecY, SecE, and SecG for 3 hours at about 37 degrees Celsius to form SecYEG impregnated proteoliposomes.

In this example, a 25-millimeter disc of hydrophilic polysulfone can be used as the membrane 602. The membrane can be soaked in a 50% ethanol solution to expand the polymer and subsequently washed in water to remove the ethanol. The membrane 602 can then be placed into the chamber 601, and a solution 606 comprising SecYEG proteoliposomes as well as additional liposomes can be deposited into the first portion 604 via the vessel 616. The solution can be incubated for 3 hours at ambient temperature to form a SecYEG impregnated supported lipid bilayer on the membrane 602. The solution 606 may be a buffer solution (e.g., pH buffered, ionic strength buffered, etc.).

After formation of the supported lipid bilayer, a flux test may be performed using a pressurized water line 607. The pressurized water line may be at a pressure of 1 bar, and the flow of water across the membrane 602 may be measured and recorded in order to determine an extent of lipid bilayer coverage of the membrane. Other examples of quality control tests include, but are not limited to fluorescence microscopy and atomic force microscopy. For example, a fluorescence microscopy image can be used to confirm the presence of the lipids of the lipid bilayer. In this example, photobleaching can be used to confirm that the lipids are a bilayer instead of immobilized unruptured liposomes. If the degree of coverage is determined to be acceptable, the chamber and membrane may be used in the formation of biological molecules.

### Example 2—Preparation of a Biological Molecule

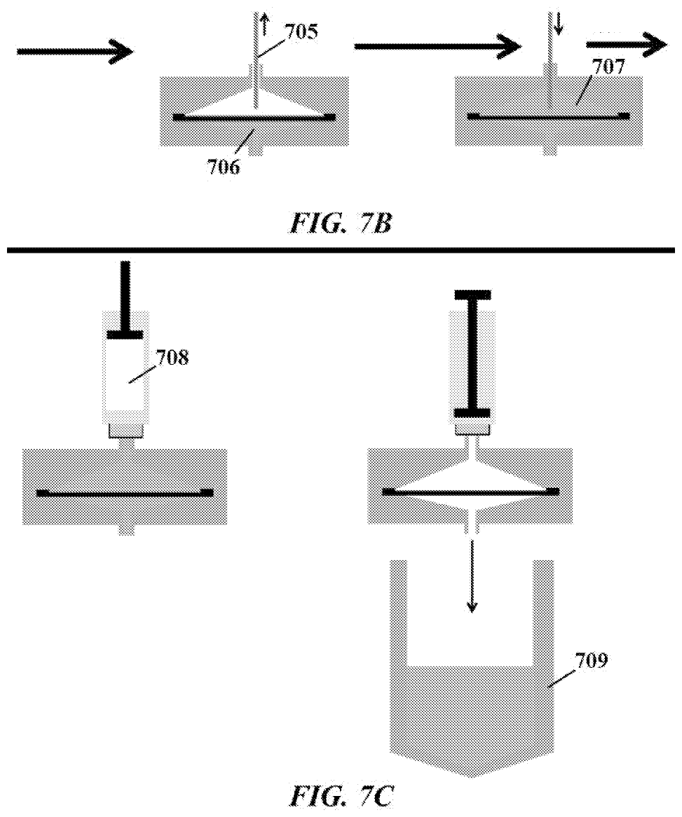

FIGS. 7A-7C are examples of a process for generating a biological molecule. Into a first portion 704 of a chamber 701 comprising a membrane 702, such as the chamber generated in Example 1, a cell-free reaction mixture 703 can be injected. The cell free reaction mixture may be generated by homogenizing E. coli cells. The lysate may be fractionated using a plurality of 12,000 rcf centrifugations to produce the cell-free reaction mixture. The mixture may be centrifuged again at 135,000 rcf to remove inverted membrane vesicles as well and can be stored at −80° C. for future use.

When the lysate 703 is added to the chamber 701, one or more nucleic acid sequences encoding for the biological molecule may be added as well. For example, DNA encoding for beta galactosidase, TrxA or OmpA can be added to form those proteins, though other proteins may be formed by similar methods. The one or more nucleic acid sequences may comprise a portion encoding for an N-term translocation signal sequence. To the lysate, additional components such as energy molecules (e.g., adenosinetriphosphate) and substrates (e.g., peptides) can be added. The chamber can be held at 37° ° C. to allow for the generation of the product biological molecule and permit the biological molecule to translocate through the membrane 702.

Subsequently to the formation and translocation of the biological molecule, the cell-free solution may be removed from the first portion 704 via a pipette or other fluid transport apparatus 705. At this point, the biological molecule can reside in the second portion 706 of the chamber 701. The first portion 704 may be rinsed one or more times to remove any additional cell-free precursors and leave a clean solution 707 in the first portion. The rise may be at a low flow rate to avoid shearing the supported lipid bilayer.

To recover the biological molecule, pressurized gas 708 (e.g., air, nitrogen, etc.) may be flushed into the chamber and rupture the supported lipid bilayer. The contents of the first and second portions may then be collected into a vessel 709 and removed. Alternatively, a fluid transport pipe can be used in place of the vessel to remove the biological molecule from the chamber.

Though described herein with respect to a single inlet and outlet chamber, the methods of the examples can be utilized in a flow cell setup. An example of a flow cell setup can be found in FIGS. 4A-4D. In a flow cell setup, both cell-free solutions as well as product biological molecules can be constantly flowed through the flow cell. An advantage of a flow cell setup is that continuous production of biological molecules can be achieved. Additionally, a flow cell setup can have improved speed of processing as well as reduced machinery costs.

### Example 3—Automated Testing of Protein Synthesis

A computer system operatively coupled to the systems described elsewhere herein can be used to provide an automated design and testing platform for biomolecule synthesis. Though described herein with respect to protein synthesis, other biological molecules as described elsewhere herein may be formed as well.

Due to the relatively short processing times of methods and systems described elsewhere herein (e.g., about 3 hours, about 5 minutes, about 30 seconds, etc.), a continuous flow system can be generated with, for example, 100 chambers each configured to produce about 0.01 mg of protein per hour for a total system rate of 1 mg per hour. The computer can determine based on analytical instruments coupled to the chambers, if each chamber of the system can (i) continue expressing the protein product of that chamber to generate additional protein for analysis or (ii) start making a different protein (e.g., a different protein entirely or a protein generated by at least one other chamber). The decision to stop (ii) may be based on having already collected enough information on the protein to know the value of continuing production of that protein. The decision in increase the number of chambers generating a particular protein can be made by determining if the protein is promising as determined by analysis performed on the chambers currently forming the protein. By directing additional chambers to form the protein, the protein can be supplied in higher quantities and/or faster.

In another example, a chamber can be configured to continually generate a protein, and the results of that synthesis can be monitored by a computer operatively coupled to the chamber. The reaction conditions of the chamber can be changed, and the effect of those changes can be tracked by the computer. In this way, the synthesis that the chamber is undertaking can be optimized in real time to produce an increase in the efficiency of that synthesis process.

### Example 4—Temporally Separated Synthesis and Collection of Biological Molecules

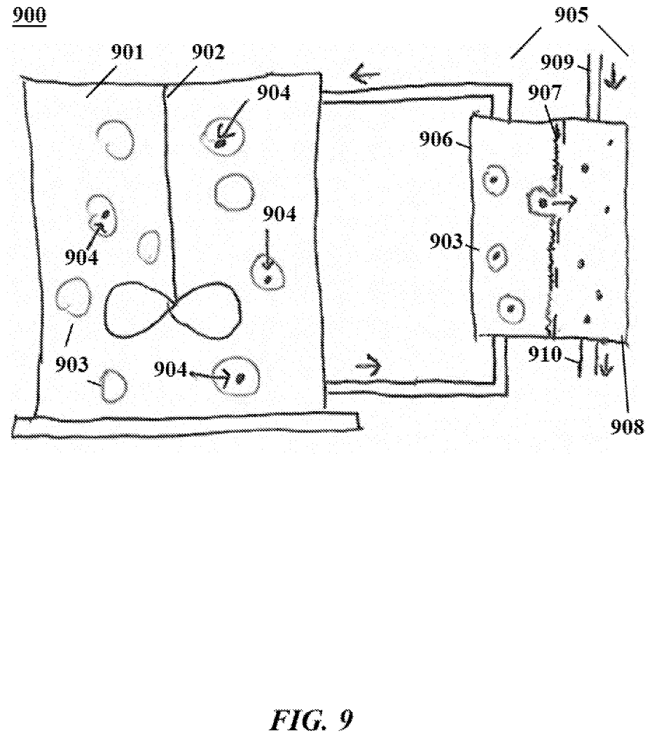

FIG. 9 shows an example of a reactor system 900 configured to produce low to no impurity biological molecules. A stir tank 901 can be configured with a stirrer 902. In some cases, the tank is not a stir tank. In some cases, the tank is a chamber as described elsewhere herein. The stir tank can be configured to stir a solution comprising a plurality of liposomes 903. The liposomes may be as described elsewhere herein. For example, the liposomes can be proteoliposomes. The liposomes may comprise translocon proteins as described elsewhere herein. For example, the liposomes can comprise SecYEG. The liposomes may be placed into a cell-free reaction mixture within the tank 901. The cell free reaction mixture may comprise precursors for a biological molecule. For example, the cell free reaction mixture can comprise DNA, amino acids, and translation molecules configured to synthesize a protein. The tank 901 comprising the cell free reaction mixture may be subjected to conditions sufficient to synthesize biological molecules 904. The biological molecules may be translocated through the translocon proteins as described elsewhere herein. The translocated biological molecules may be contained within the liposomes 903. The translocated biological molecules may be substantially free of impurities. For example, the interior of the liposomes may be free from any of the cell free precursors. In this example, the biological molecules may be pure. The solution comprising the liposomes 903 may be transported to a chamber 905 comprising a first portion 906, a membrane 907, and a second portion 908. The first portion, second portion, and membrane may be as described elsewhere herein. For example, the membrane can be a supported lipid bilayer. In another example, the membrane may not comprise any translocon proteins. The liposomes may be configured to fuse with the membrane 907 in order to deliver the biological molecule 904 from the first portion 906 to the second portion 908. When the liposomes fuse with the membrane, the biological molecule can flow from the liposome into the second portion, where a flow of solution from inlet 909 to outlet 910 can extract the biological molecule. The process of fusing the liposomes to the membrane may be a fast process. As such, the biological molecules may be rapidly extracted from the first portion to the second portion. The conditions of the second portion (e.g., presence or absence of chaperones, folding conditions, etc.) can be as described elsewhere herein. The conditions of the second portion may be the same as the conditions within the liposomes 904. For example, the interior of the liposomes can comprise chaperones and a solution configured to aid in the folding of the biological molecule. In this example, the same solution can be present in the second portion, and the chaperones can be present in the membrane. In this example, the liposomes can be generated in a solution that can be the same as the solution in the second portion (e.g., the liposomes can be prepared with a same buffer as the second portion). In some cases, liposomes can be recycled out of the membrane. For example, clathrin can be used to bud proteoliposomes back out of the membrane, which can be recirculated back into the tank and reused.

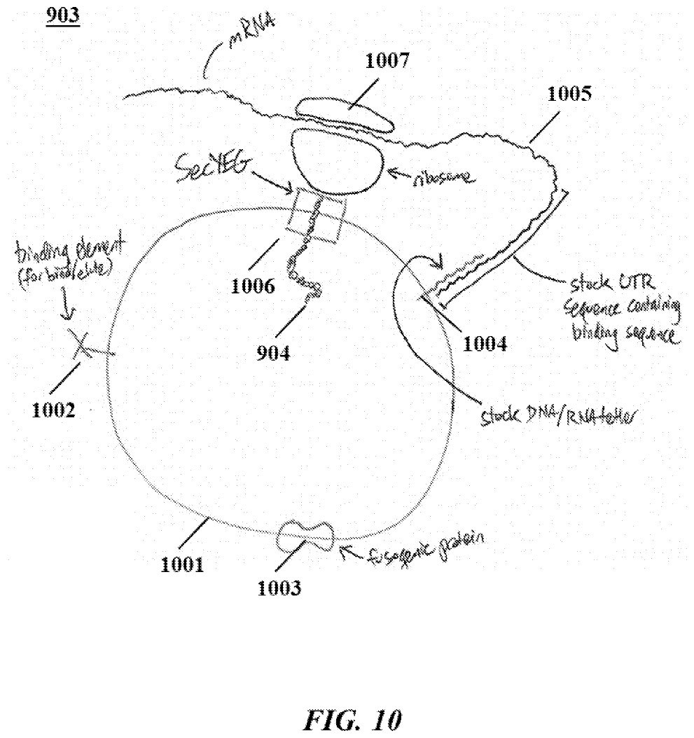

FIG. 10 shows an example of a liposomes 903 configured for the production of a biological molecule. A lipid bilayer 1001 can comprise a binding element 1002. The binding element may be as described elsewhere herein. For example, the binding element can be configured to maintain the liposome adjacent to a support (e.g., a reactor wall, a bead, etc.). For example, the binding element can be configured to prevent the liposome from exiting the tank before the biological molecule is formed. The binding element may comprise an oligonucleotide, an antibody, an antigen, a protein, a chelating agent, a metal ion, or the like, or any combination thereof. The binding agent may permit reversable binding of the liposome to a support. The liposome may comprise a fusogenic portion 1003. Examples of fusogenic portions include, but are not limited to fusion-associated small transmembrane (FAST) proteins, boronic acid or inositol (e.g., the liposome has either boronic acid or inositol and the membrane has the other), izumol or juno (e.g., the liposome has either izumol or juno and the membrane has the other), or the like, or any combination thereof. The use of fusogenic portions that are complementary to other fusogens (e.g., boronic acid/inositol, izumol/juno, etc.) may promote fusion of the liposome to the predetermined target (e.g., the membrane, liposomes comprising reagents, etc.) and not other liposomes. The fusogenic portion may be configured to aid in the fusing of the liposome to the membrane. For example, the fusogenic portion can be a protein configured to promote fusion of the liposome's lipid bilayer with that of the membrane. The fusogenic portion may comprise a protein, an organic molecule (e.g., ethylene glycol, an organic surfactant, etc.), or the like, or any combination thereof. The liposome may comprise a first oligonucleotide 1004. The first oligonucleotide 1004 may be tethered to the liposome. For example, the first oligonucleotide may be embedded in the liposome. In another example, the first oligonucleotide can be attached to a base that is in turn embedded in the liposome. The first oligonucleotide 1004 can be configured to bind to at least a portion of a second oligonucleotide 1005. The second oligonucleotide can comprise a portion that encodes for the biological molecule. For example, the second oligonucleotide can comprise an mRNA sequence for a protein biological molecule. The second oligonucleotide may be of sufficient length to permit a ribosome to interact with the liposome (e.g., be positioned above a translocon protein). For example, the second oligonucleotide can be engineered with extra bases to avoid steric hinderance of the ribosome interacting with the liposome. In another example, the second oligonucleotide can be natively long enough to avoid steric hinderances. The first and second oligonucleotides may be different for different liposomes in the tank. For example, a tank comprising two different sets of first and second oligonucleotides can be configured to generate two different biological molecules corresponding to the different sets. In this example, the reactor system can output a mixture of the two biological molecules. In another example, the different oligonucleotides can be tags corresponding to different conditions within the different liposomes (e.g., different folding conditions, buffers, etc.). In this example, different conditions can be tested for a same biological molecule in a parallel way to provide the optimal conditions for a given biological molecule. The oligonucleotide used for identification of the liposomes and related properties (e.g., identity of the formed biological molecule, conditions, etc.) can be a same oligonucleotide as is used to bind the second oligonucleotide. Alternatively, the oligonucleotide can be a separate oligonucleotide configured for use as an identification oligonucleotide.

The liposome may comprise a translocon protein 1006. The translocon protein may be as described elsewhere herein. For example, the translocon protein can be SecYEG. The translocon protein may be adjacent to a ribosome 1007. The ribosome may be configured to translate the second oligonucleotide 1005 to form the biological molecule 904, which can translocate into the liposome through the translocon protein 1006.

The biological molecule can then be collected. Alternatively, additional liposomes comprising analytical reagents can be introduced to the liposome. The analytical reagents can then perform in situ characterization of the biological molecule. Alternatively, the analytical reagents can prepare the biological molecule for flow cytometry. In an example, a plurality of oligonucleotide template can be introduced into a reactor comprising a plurality of liposomes. In this example, the oligonucleotides can bind to the liposomes and be used to generate a plurality of biological molecules. In this example, once the biological molecules are formed, liposomes comprising analytical reagents can be introduced to the liposomes with the biological molecules. In this example, the analytical reagents can identify the liposomes which comprise a biological molecule of interest, and those liposomes can be sorted by flow cytometry and the oligonucleotides bound to the liposomes can be sequenced. Such a scheme can provide for fast and simple discovery processes.

A liposome based biological molecule reactor can be used in a directed evolution/mutagenesis scheme. Liposomes that contain the biological molecule can be sorted by flow cytometry and the biological molecules can be used as inputs for a reverse transcription process. The reverse transcribed oligonucleotide can then be used as an input for a transcription process. Either or both of the reverse transcription process and the transcription process may be error-prone (e.g., subject to a high likelihood of mutation). The new biological molecule variants can then be tested for a given property (e.g., binding affinity), and the oligonucleotide sequence that gave rise to that new biological molecule can be sequenced. In this way, a plurality of different sequences for a plurality of biological molecule variants can be quickly determined and screened.

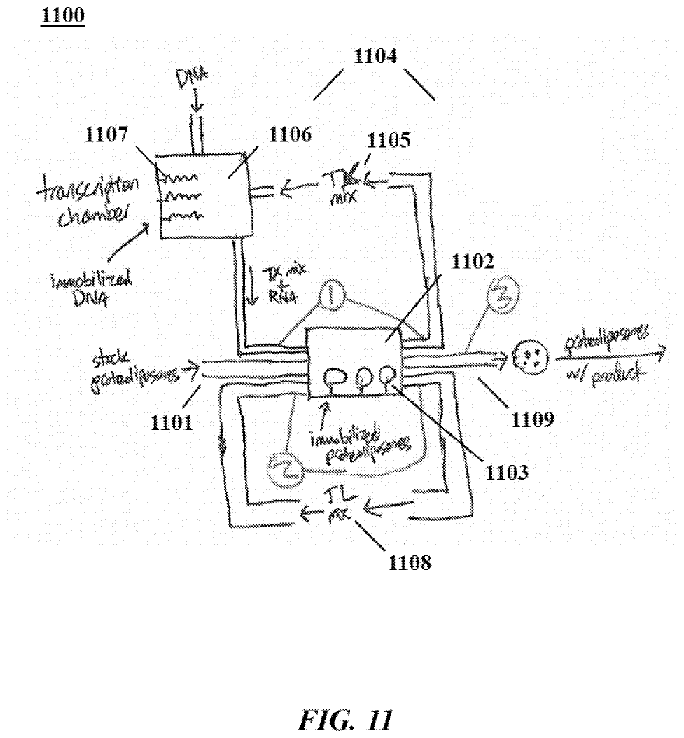

FIG. 11 shows an example of a discovery platform 1100. Proteoliposomes (e.g., liposome 904 of FIG. 10) can be introduced via flow path 1101 into chamber 1102. The chamber may be configured with binding sites configured to interact with a binding element of the proteoliposome. The proteoliposome can then bind to the walls of the chamber to form bound liposomes 1103. The chamber may comprise a resin configured to bind the proteoliposomes. The presence of a resin may provide an increased surface are for binding the proteoliposomes. In another portion of the platform 1104, a transcription reagent mixture 1105 can be flowed into a second chamber 1106. The second chamber can comprise immobilized oligonucleotides 1107. The immobilized oligonucleotides can be immobilized DNA corresponding to a predetermined biological molecule. The immobilized oligonucleotides may be immobilized DNA corresponding to a plurality of biological molecules. The transcription reagent mixture can comprise reagents to transcribe the immobilized oligonucleotides to form unbound oligonucleotides (e.g., mRNA). The unbound oligonucleotides can be flowed into the chamber 1102 and attached to the surface of the proteoliposomes 1103. After attachment, the excess transcription reagents and unbound oligonucleotides can be removed from the chamber 1102. The removal of the excess transcription reagents may be a removal without impurities. For example, the transcription reagents can be removed without impurities from the proteoliposomes. In this example, the pure transcription reagents may be reused. Before being recycled, the transcription reagents can be flowed through a chamber comprising a plurality of filter oligonucleotides. The filter oligonucleotides can be the same sequence as the first oligonucleotide in the liposome. The presence of the filter oligonucleotides can remove any second oligonucleotide impurity from the transcription reagents. By removing any second oligonucleotide impurities, the transcription reagents can be reused for generating a second biological molecule without any biological molecule byproducts generated by the presence of the second oligonucleotide. The filter oligonucleotides can be attached to a resin to increase surface area. Subsequently, a translation reagent mixture 1108 can be flowed into the chamber 1102. The translation reagent mixture can comprise reagents sufficient to translate the nucleotides attached to the liposomes to form biological molecules. The biological molecules can be formed directly into the liposomes as shown in FIG. 10. Subsequent to the forming of the biological molecules, the translation reagent mixture can be removed from the chamber 1102. The removal may be a removal of the translation reagents without impurities. For example, the translation reagent mixture can be removed without impurities from the biological molecules. In this example, the translation reagent mixture can be reused. Subsequently to the removal of the translation reagent mixture, the proteoliposomes 1103, which can now comprise the biological molecules, can be eluted out of the chamber via flow path 1109. The eluting may comprise disruption of a reversable bond between the proteoliposome and the side of the chamber. The proteoliposomes can be fused to a membrane as described elsewhere herein to extract the biological molecules without impurities. The proteoliposomes may be introduced to a plurality of membranes. For example, a chamber comprising three membranes can be used to improve throughput. The proteoliposomes may be fused with secondary liposomes containing analytical reagents (e.g., protein sequencing reagents, etc.) and analyzed. The analysis may comprise use of flow cytometry. For example, for a plurality of proteoliposomes with different oligonucleotides and biological molecules, the biological molecules of interest can be identified, and the associated oligonucleotides can be sequenced.

### Example 5—Quality Control of Proteoliposomes

A quality control scheme for a proteoliposome generating procedure can improve product quality and permit faster development of new proteoliposome systems. Such a scheme may comprise use of a full transcription, translation, and translocation assay. In some cases, a shorter scheme can be used to provide improved turnaround times.

A peptide probe can be translated through the proteoliposome for a quality control scheme. The peptide probe can be configured to conjugate to a fluorophore (e.g., fluorescein) (e.g., by comprising cysteine residues). The peptide probe may be synthesized in a separate reaction from the production of the proteoliposome to be quality controlled. For example, the peptide probe can be synthesized and subsequently introduced to the proteoliposome. The peptide probe may not be purified from the reaction mixture used to generate the peptide probe. For example, the peptide probe can be kept in the raw reaction mixture when it is supplied to the proteoliposome. Supplying the peptide probe in the reaction mixture can simplify the process by removing a purification process from the quality control process. Another benefit of an already made peptide probe may be that the quality control translocation reactions can be executed and analyzed faster than a quality control reaction comprising synthesis of the peptide probe. Another benefit may be a simplification of the instrument used for the quality control testing. For example, after processing to remove the proteoliposomes from peptide probes and fluorophores in the solution (e.g., by centrifugation, ultracentrifugation, sucrose gradient density ultracentrifugation, size exclusion chromatography, trichloroacetic acid precipitation, etc.), a fluorescence measurement may be sufficient for the quality control procedure (e.g., the fluorescence measurement may be sufficient to provide information about the quality of the proteoliposomes). In some cases, the peptide probe can be purified from the reaction mixture. The reaction mixture comprising the peptide probe can comprise adenosine triphosphate (ATP), SecB, SecA, buffer, or the like, or any combination thereof.

In some cases, the conditions in the interior of the proteoliposome can be such that the peptide probe undergoes a change upon translocation into the proteoliposome. For example, the peptide probe within the proteoliposome can interact with agents configured to alter the fluorescence of the mixture of translocated and non-translocated peptide probes. For example, a fluorescence quencher can be placed inside the proteoliposomes such that, upon translation, the total fluoresce of the mixture can be decreased. In this example, the amount of decrease of the overall fluorescence can be directly related to how much of the peptide probes have translocated. Incorporating such changes to the interior of the proteoliposomes can increase the speed of the quality control process (e.g., by removing a purification operation between translocation and analysis).

### Example 6—Droplet Based Proteoliposome Platform

A droplet based microfluidic platform may be combined with the proteoliposome platforms described elsewhere herein. This may provide enhanced control over the expression of nucleic acids to produce biological molecules, and provide enhanced colocalization of genotype/phenotype expression. The droplet based microfluidic platform may be configured to generate a plurality of droplets within the microfluidic platform (e.g., by generating water droplets suspended in immiscible oil, etc.).

In some cases, the individual droplets of the microfluidic platform can be configured as individual one-pot reaction vessels. For example, each droplet of the microfluidic platform can be configured to comprise one-pot reaction mixtures as described elsewhere herein. In some cases, each droplet can comprise at most a single nucleic acid such that a single species of biological molecule is generated per droplet. For example, the droplet can be generated, and at most about one nucleic acid molecule can be added to the droplet. The single nucleic acid molecule may correspond to a single biological molecule type being produced in each individual droplet. In some cases, the nucleic acid may be tethered to the proteoliposome. In some cases, the droplet can comprise a tag configured to provide information about the conditions within the proteoliposome (e.g., folding reagents, etc.) and/or identity of the nucleic acid. For example, a DNA tag can be included in the proteoliposome to identify the folding conditions within the proteoliposome. In this way, the conditions may be tracked across the different droplets within the microfluidic system. In some cases, the proteoliposomes used in the microfluidic system may be large (e.g., greater than about 500 nanometers in diameter) to improve the amount of biological molecule that can be taken into the proteoliposome, thereby decreasing the risk of aggregation of the biological molecule. In some cases, a greater number of proteoliposomes can be included in the droplet to reduce the risk of aggregation, as the resultant biological molecules can be divided into the increased number of proteoliposomes.

### Example 7—Proteoliposome Based Packaging of Biomolecules

A biological molecule translocated into a proteoliposome may be in a stable environment for transport. For example, the proteoliposome may provide conditions configured to keep the biological molecule safe during transport. The encapsulated biological molecule may be stable for a longer time than a biological molecule that is not encapsulated, be more temperature stable, be more stable at higher temperatures (e.g., 4 degrees Celsius, room temperature, etc.), have reduced protease sensitivity, and the like, as compared to a biological molecule that is not encapsulated. The biological molecule may be later removed from the proteoliposome as described elsewhere herein.

Additionally, a proteoliposome can serve as a delivery vehicle for a biological molecule. For example, the biological molecule that was translocated into the proteoliposome can be a drug molecule, and the translocation can purify the biological molecule and place it in a liposome delivery vehicle. In some cases, the proteoliposome can be configured with additional elements configured to improve its performance as a drug delivery vehicle, including, but not limited to, targeting compounds, immunogenicity suppressants (e.g., polyethylene glycol), etc.

While preferred embodiments of the present invention have been shown and described herein, it will be obvious to those skilled in the art that such embodiments are provided by way of example only. It is not intended that the invention be limited by the specific examples provided within the specification. While the invention has been described with reference to the aforementioned specification, the descriptions and illustrations of the embodiments herein are not meant to be construed in a limiting sense. Numerous variations, changes, and substitutions will now occur to those skilled in the art without departing from the invention. Furthermore, it shall be understood that all aspects of the invention are not limited to the specific depictions, configurations or relative proportions set forth herein which depend upon a variety of conditions and variables. It should be understood that various alternatives to the embodiments of the invention described herein may be employed in practicing the invention. It is therefore contemplated that the invention shall also cover any such alternatives, modifications, variations, or equivalents. It is intended that the following claims define the scope of the invention and that methods and structures within the scope of these claims and their equivalents be covered thereby.

What is claimed is:

1. A method for generating a biological molecule, comprising:
(a) providing a chamber comprising a plurality of cell-free precursors of said biological molecule and a proteoliposome, wherein said proteoliposome comprises a pore;
(b) using at least a subset of said plurality of cell-free precursors to form said biological molecule; and
(c) during or subsequent to (b), translocating at least a portion of said biological molecule through said pore into said proteoliposome.

2. The method of claim 1, wherein said proteoliposome comprises a lipid bilayer.

3. The method of claim 2, wherein said lipid bilayer comprises one or more translocon proteins.

4. The method of claim 1, further comprising (d) removing said proteoliposome from said chamber.

5. The method of claim 1, wherein said biological molecule further comprises an N-terminal translocation signal sequence.

6. The method of claim 5, wherein, subsequent to (c), said N-terminal translocation signal sequence is removed from said biological molecule.

7. The method of claim 1, wherein said translocating occurs substantially simultaneously to said forming said biological molecule.

8. The method of claim 1, wherein said translocating occurs subsequently to said forming said biological molecule.

9. The method of claim 1, wherein said translocating occurs co-translationally.

10. The method of claim 1, wherein said biological molecule is a polypeptide.

11. The method of claim 10, wherein said polypeptide is a protein, and wherein at least a portion of said protein is formed in said first portion and folded in said second portion.

12. The method of claim 1, wherein said chamber is a part of a flow channel.

13. The method of claim 1, wherein said cell-free precursors do not comprise said biological molecule.

14. The method of claim 1, wherein (c) comprises translocating an entirety of said biological molecule through sad pore and into said proteoliposome subsequent to (b).

15. The method of claim 1, wherein (c) is performed during (b).

16. The method of claim 1, wherein (c) is performed subsequent to (b).

17. A system for generating a biological molecule, comprising:
a chamber comprising
a first portion configured to comprise a plurality of cell-free precursors of said biological molecule and a proteoliposome comprising one or more translocon proteins.

18. The system of claim 17, wherein said one or more translocon proteins comprise one or more proteins selected from the group consisting of SecYEG, SecY, SecE, SecG, Sec61p, and an injectosome.

19. The system of claim 17, wherein said plurality of cell-free precursors do not comprise one or more cells.

20. The system of claim 17, wherein said plurality of cell-free precursors comprises deoxyribonucleic acid (DNA).

21. The system of claim 20, wherein said DNA encodes for said biological molecule.

22. The system of claim 17, wherein said biological molecule is a protein, and wherein said proteoliposome comprises conditions for optimal folding of said protein.

23. The system of claim 17, wherein said biological molecule is a nucleic acid molecule, a protein, an antigen, a polypeptide, an enzyme, or a chemical.

24. The system of claim 17, wherein said proteoliposome comprises one or more signal peptidase proteins.

25. A method for generating a polypeptide, comprising
(a) using a cell-free solution comprising a deoxyribonucleic acid molecule encoding said polypeptide to generate a ribonucleic acid molecule,
(b) using said ribonucleic acid molecule to generate said polypeptide, and
(c) directing said polypeptide through a pore disposed in a proteoliposome.

26. The method of claim 25, wherein, subsequent to (c), said polypeptide is present at a purity of at least 60%.

27. The method of claim 25, wherein (a)-(c) is performed in a time period of at most 1 day.

28. The method of claim 25, wherein said polypeptide comprises a non-native N-terminal signal sequence.

29. The method of claim 25, wherein said proteoliposome comprises one or more translocon proteins.

30. The method of claim 25, wherein said proteoliposome comprises one or more signal peptidase proteins.

31. The method of claim 25, wherein said polypeptide is a protein.
